(19) United States
(12) Patent Application Publication
(10) Pub. No.: US 2020/0292565 A1
(43) Pub. Date: Sep. 17, 2020

(54) AUTOMATED NUCLEIC ACID SAMPLE PREPARATION, DETECTION, AND ANALYSIS SYSTEM

(71) Applicant: Shanghai Rendu Biotechnology Co., Ltd., Shangahi (CN)

(72) Inventors: Jinliang JU, Shanghai (CN); Hanwu ZHENG, Shanghai (CN); Yachen MAO, Shanghai (CN); Hao JIN, Shanghai (CN); YI CHEN, Shanghai (CN)

(73) Assignee: Shanghai Rendu Biotechnology Co., Ltd., Shangahi (CN)

(21) Appl. No.: 16/755,050

(22) PCT Filed: Oct. 11, 2017
(86) PCT No.: PCT/CN2017/105631

§ 371 Date: Apr. 9, 2020

(51) Int. Cl. G01N 35/00; C12N 15/10; G01N 35/10; B01L 3/02; B01L 9/06; B01L 3/00; C12Q 1/6806; C12Q 1/6851

(52) U.S. Cl. CPC G01N 35/0099; C12N 15/10; G01N 35/10; B01L 3/021; B01L 2300/025; B01L 3/5082; C12Q 1/6806; C12Q 1/6851; B01L 2200/143; B01L 9/06

(57) ABSTRACT

Described herein is an integrated automated nucleic acid sample preparation, detection and analysis system, along with methods of operating such a system. Also described are methods of methods of analyzing nucleic acid molecules in a sample and methods of determining a melting curve of a nucleic acid sample. The automated system includes a robotic pipettor comprising one or more pipettes movable in a horizontal plane and configured to dispense or withdraw one or more liquids, a robotic arm configured to transport a plurality of connected sample processing tubes, a nucleic acid isolation system comprising a first sample processing tube holder configured to hold the plurality of connected sample processing tubes, and magnet, wherein magnetically-responsive particles contained within each sample processing tube are retained within the sample processing tube upon withdrawing of liquid by the robotic pipettor when the magnet is in an active configuration; and a fluorometer comprising a light source and an optical detector disposed below a second sample processing tube holder configured to hold the plurality of connected sample processing tubes, the second sample processing tube holder having a clear or open bottom, wherein the fluorometer is configured to detect fluorescence emitted from a sample in one or more of the sample processing tubes.

### TECHNICAL FIELD

The present invention relates to a system for the automated sample preparation, nucleic acid detection and analysis of biological samples.

### BACKGROUND

Automated systems for the preparation of biological samples provide increased efficiency and quality control compared to manual sample preparation. The QIAsympohany® SP from QIAGEN, for example, is an automated system for preparing nucleic acid samples. The prepared and purified nucleic acid samples can then be transported from the sample preparation system for sample detection and analysis.

Designing fully integrated systems for efficiently preparing and analyzing biological samples in a confined space with tight quality control is substantially more complex. Moving components of the system must be carefully designed to avoid cross contamination. Further, liquid and solid waste management allows for continuous operation of the automated system. What is needed in the art is a compact, automated system to efficiently prepare and analyze nucleic acid samples with optimized solid and liquid waste management.

The disclosures of all publications, patents, and patent applications referred to herein are hereby incorporated herein by reference in their entireties.

### SUMMARY OF THE INVENTION

Described herein is an integrated automated nucleic acid isolation and analysis system, methods of operating the system, methods of analyzing nucleic acid molecules in a sample, and methods of determining a melting curve of a nucleic acid sample.

In one aspect, the automated nucleic acid isolation and analysis system comprises a robotic pipettor comprising one or more pipettes movable in a horizontal plane and configured to dispense or withdraw one or more liquids; a robotic arm configured to transport a plurality of connected sample processing tubes; a nucleic acid isolation system comprising a first sample processing tube holder configured to hold the plurality of connected sample processing tubes, and magnet; wherein magnetically-responsive particles contained within each sample processing tube are retained within the sample processing tube upon withdrawing of liquid by the robotic pipettor when the magnet is in an active configuration; and a fluorometer comprising a light source and an optical detector disposed below a second sample processing tube holder configured to hold the plurality of connected sample processing tubes, the second sample processing tube holder having a clear or open bottom, wherein the fluorometer is configured to detect fluorescence emitted from a sample in one or more of the sample processing tubes.

In some embodiments, the fluorometer is configured to heat the plurality of connected sample processing tubes to a predetermined temperature above room temperature.

In some embodiments, the plurality of connected sample processing tubes is a sample strip comprising three or more linearly arranged sample processing tubes. In some embodiments, the plurality of connected sample processing tubes is a multi-well plate.

In some embodiments, the system comprises a heated vessel comprising wax, wherein the wax is heated by the vessel to a temperature above the melting temperature of the wax.

In some embodiments, the system comprises one or more heated incubators configured to heat the plurality of connected sample processing tubes.

In some embodiments, the system comprises one or more shakers configured to vortex a sample contained within the sample processing tubes.

In some embodiments, the system comprises a sample source tube holder configured to hold the plurality of sample source tubes.

In some embodiments, the system comprises a barcode scanner configured to read a sample barcode disposed on one or more sample source tubes or the plurality of sample processing tubes.

In some embodiments, the system comprises a pipette tip holder accessible by the plurality of pipettes.

In some embodiments, the system comprises a reagent rack configured to hold one or more reagents.

In some embodiments, the system comprises a solid waste management system configured to receive pipette tips and the plurality of sample processing tubes.

In some embodiments, the system comprises one or more cooling racks configured to hold the sample processing tube.

In some embodiments, the system comprises a liquid waste management system comprising a liquid waste port and a conduit configured to drain the liquid waste from the liquid waste port.

In some embodiments, the robotic pipettor is operable to move the plurality of pipettes in a predetermined path that prevents the plurality of pipettes from moving above a non-targeted system component.

In some embodiments, the system comprises a housing enclosing the system, the housing comprising a base and an openable lid. In some embodiments, the housing comprises a ventilation system comprising an air filter, wherein the ventilation system is configured to provide filtered air to the enclosed system and withdraw air from the enclosed system. In some embodiments, the enclosed system is operated at a higher pressure than the pressure external to the housing. In some embodiments, the housing comprises one or more indicator lights on an external surface of the housing configured to indicate normal operation of the system or an error. In some embodiments, the system comprises a UV light within the housing configured to sterilize the system when the UV light is operated.

In some embodiments, the system comprises an indicator to indicate an error. In some embodiments, the indicator is a light or an audible alarm.

In some embodiments, the system comprises a computer system for operating the automated nucleic acid isolation and analysis system. In some embodiments, the computer system comprises a display. In some embodiments, the computer system is connected to a laboratory information system configured to store or transmit sample analysis results.

In another aspect, there is a method of analyzing nucleic acid molecules in a sample, comprising isolating nucleic acid molecules comprising a region of interest from the sample; combining the nucleic acid molecules, a nucleic acid probe that hybridizes to at least a portion of the region of interest, and a fluorophore; adding a melted wax with a melting temperature above room temperature to a sample processing tube containing the sample; amplifying the region of interest; measuring fluorescence of the combined region of interest, the nucleic acid detection probe, and the fluorophore; solidifying the wax in the sample processing tube; and discarding the sample processing tube comprising the solidified wax. In some embodiments, the method comprises determining an amplification curve for the sample. In some embodiments, the fluorophore is attached to the nucleic acid probe. In some embodiments, the fluorophore is separate from the nucleic acid probe.

In another aspect, there is a method of determining a melting curve of a nucleic acid sample, comprising isolating nucleic acid molecules comprising a region of interest from the sample; combining the nucleic acid molecules, a nucleic acid probe that hybridizes to at least a portion of the region of interest, and a fluorophore; adding a melted wax with a melting temperature above room temperature to a sample processing tube containing the sample; amplifying the region of interest from the nucleic acid molecules; measuring fluorescence of the combined region of interest, the nucleic acid detection probe, and fluorophore at a plurality of temperatures; solidifying the wax contained within the sample processing tube; and discarding the sample processing tube comprising the solidified wax.

In some embodiments of the above methods, the fluorophore is separate from the nucleic acid probe.

In some embodiments of the above methods, the method is performed by an automated system.

In some embodiments of the above methods, the sample processing tube is passively cooled.

In some embodiments of the above methods, the method comprises heating the sample processing tube to denature the region of interest and the nucleic acid detection probe.

In some embodiments of the above methods, isolating the nucleic acid comprises binding magnetically-responsive particles functionalized with a nucleic acid capture probe to the nucleic acid molecules comprising the region of interest.

In some embodiments of the above methods, the method comprises washing the magnetically-responsive particles bound to the nucleic acid molecules comprising the region of interest.

In some embodiments of the above methods, isolating the nucleic acid molecules comprises lysing cells containing the nucleic acid molecules.

In some embodiments of the above methods, fluorescence is measured from below the sample processing tube.

In some embodiments of the above methods, the method further comprises analyzing the measured fluorescence to determine an amount of the region of interest in the sample.

In some embodiments of the above methods, the melting temperature the wax is about 30° C. to about 90° C. In some embodiments, the wax is paraffin.

In another aspect, there is a method of analyzing a nucleic acid sample, comprising: dispensing a sample comprising nucleic acid molecules comprising a region of interest into a sample processing tube selected from a plurality of connected sample processing tubes; combining the sample with magnetically-responsive particles functionalized with a probe that binds to the nucleic acid molecules comprising the region of interest; transporting the sample processing tube using a robotic arm to a magnetic module comprising a first sample holder configured to hold the plurality of connected sample processing tubes, and magnet; washing the nucleic acid molecules using a robotic pipettor by dispensing and withdrawing a wash buffer into the sample processing tube, wherein the magnet is in an active configuration when the wash buffer is withdrawn, thereby retaining the magnetically-responsive particles in the sample processing tube; adding a melted wax the sample processing tube using the robotic pipettor, wherein the wax has a melting temperature above room temperature to the sample processing tube; adding an amplification reagent, a nucleic acid probe that specifically binds to the nucleic acid molecules, and a fluorophore to the sample processing tube using the robotic pipettor; transporting the sample processing tube to a second sample holder on a fluorometer using the robotic arm, wherein the second sample holder is disposed above a light source and an optical detector; simultaneously heating the sample processing tube and detecting fluorescence from the sample; cooling the sample processing tube, thereby solidifying the wax; and discarding the sample processing tube comprising the solidified wax.

### BRIEF DESCRIPTION OF THE FIGURES

FIG. 1 illustrates an overhead schematic of an exemplary automated system for biological cell lysis, nucleic acids capture and isolation, nucleic acid amplification, and nucleic acid analysis. The robotic pipettor and the robotic arm configured to transport sample processing tubes is removed from this figure for easier viewing.

FIG. 2 illustrates an exemplary automated system for isolating and analyzing nucleic acids enclosed in a housing. The system includes a display for a computer system, which is external to the housing.

FIG. 3A-C illustrates a strip of six linearly arranged, connected sample processing tubes. FIG. 3A shows a front view, FIG. 3B shows a side view, and FIG. 3C shows a top view of the stip.

FIG. 4A shows an exemplary sample source tube holder with a barcode scanner attached to the side of the sample source tube holder. The sample source tube holder illustrated in FIG. 4A includes a plurality of slots for sample source tubes, configured in 20 columns and 8 rows. FIG. 4B shows a side view of one of the rows of slots.

FIG. 5 illustrates an exemplary sample processing tube holder.

FIG. 6 illustrates an exemplary pipette tip holder, which can receive cartridges of pipette tips.

FIG. 7 illustrates an exploded view of an exemplary embodiment of a solid waste management system.

FIG. 8A illustrates one embodiment of a reagent rack. FIG. 8B illustrates another embodiment of a reagent rack. In certain embodiment, the automated system includes two or more different types of reagent rack, such as the reagent rack shown in FIG. 8A and the reagent rack shown in FIG. 8B.

FIG. 9A shows a shaker with a sample processing tube holder configured to engage the shaking platform of the shaker. FIG. 9B shows the shaker without the sample processing tube holder, and FIG. 9C shows the sample processing tube holder configured to be used with the shaker.

FIG. 10A illustrates an exploded view of an exemplary heated incubator with a sample processing tube holder. FIG. 10B shows a vertical cross-section of the exemplary assembled heated incubator.

FIG. 11 illustrates an exploded view of an exemplary nucleic acid isolation system, including a sample processing tube holder and a plurality of magnets.

FIG. 12 illustrates an exemplary liquid waste port that can be used with the liquid waste management system.

FIG. 13 illustrates an exploded view of an exemplary nucleic acid amplification and detection system with a fluorometer that can be used with the automated system.

FIG. 14 illustrates a schematic of a computer system that can be used to operate the automated system.

### DETAILED DESCRIPTION OF THE INVENTION

Described herein is an integrated system for automated sample preparation and analysis of a nucleic acid sample. Also described herein are methods of analyzing a nucleic acid sample, as well as methods of determining a melting curve of a nucleic acid sample. The methods may be performed, for example, using the automated nucleic acid preparation and analysis system.

The system is configured to receive biological samples (such as blood, plasma, saliva, solid tissue, semen, sputum, or urine) and isolate nucleic acid within the biological sample. In some embodiments, nucleic acid isolation is performed, at least in part, using a magnetic module that can retain magnetic beads bound to the nucleic acid molecules during nucleic acid isolation. The system can combine the isolated nucleic acid molecules with a nucleic acid probe and a fluorophore. In some embodiments, the nucleic acid probe hybridizes to a target region of interest, and the fluorophore can intercalate the resulting double stranded nucleic acid. In some embodiments, the system includes a fluorometer configured to detect fluorescence emitted from a sample in one or more sample processing tubes. The fluorometer can be configured to heat the one or more sample processing tubes to a temperature above room temperature, for example for isothermal amplification and/or determining a melting curve.

The automated system can further include one or more robotic pipettors comprising one or more pipettes. In some embodiments, the robotic pipettor consists of a single pipette, which can help limit cross-contamination in the system, as further explained below. The robotic pipettor is configured to move the one or more pipettes in a horizontal plane within the system, which allows the system to dispense or withdraw one or more liquids from or to locations within the system. The robotic pipettor can also move in vertical axis, which can help enhance accuracy of pipetting and/or replace pipette tips.

The automated system can also include a robotic arm configured to transport one or more sample processing tubes within the system. In some embodiments, a plurality of sample processing tubes are connected (such as in a linear strip or a plate), and the robotic arm can transport the plurality of sample processing tubes.

In some embodiments, the system includes a heated vessel that contains a melted wax. The wax (such as paraffin) melts at a temperature above room temperature, and is a solid at room temperature. The system can be configured to add melted wax to a sample processing tube, which sits on top of the sample in the sample processing tubes. The wax acts to limit evaporation of liquids within the sample processing tube during the sample processing and/or analysis stages, The wax is also a solid at room temperature, which traps the liquids in the sample processing tube. Thus, the wax facilitates disposal of used sample processing tubes by allowing the sample processing tubes to be disposed of in a solid waste container without risk of spillage or leakage of liquids. This enhances safety by limiting risk of biohazardous liquid spills, and allows for easier waste management.

In some embodiments, an automated sample preparation, nucleic acid isolation, and nucleic analysis system comprises a robotic pipettor comprising one or more pipettes movable in the a horizontal plane and configured to dispense or withdraw one or more liquids; a robotic arm configured to transport a plurality of connected sample processing tubes; a magnetic module comprising a first sample holder configured to hold the plurality of connected sample processing tubes, and magnet; wherein magnetically-responsive particles contained within each sample processing tube are retained within the sample processing tube upon withdrawing of liquid by the robotic pipettor when the magnet is in an active configuration; and a fluorometer comprising a light source and an optical detector disposed below a second sample holder configured to hold the plurality of connected sample processing tubes, the second sample holder having a clear or open bottom, wherein the fluorometer is configured to detect fluorescence emitted from a sample in one or more of the sample processing tubes.

Also described herein is a method of analyzing nucleic acid molecules in a sample, which can be performed by an automated system. The method includes isolating nucleic acid molecules comprising a region of interest from the sample, for example by using a magnetic module. The isolated nucleic acid molecules are combined with a nucleic acid probe that hybridizes to the region of interest and a fluorophore. Melted wax is then added to the sample in a sample processing tube, which floats on top of the sample. The nucleic acid molecules in the sample can be amplified, for example by adding polymerase to the sample which may or may not already have wax on top of the sample). Amplification can occur, for example, under isothermal conditions. In some embodiments, fluorescence of the sample is measured, which may occur simultaneously during amplification (i.e., at a plurality of time points to obtain an amplification curve) or after amplification (i.e., to obtain an end point fluorescence). The wax is solidified, and the sample processing tube containing the sample and the solidified wax is then discarded.

In another aspect, there is a method of determining a melting curve of a nucleic acid sample, which can be performed by the automated system. The method includes isolating nucleic acid molecules comprising a region of interest from the sample, for example by using a magnetic module. The isolated nucleic acid molecules are combined with a nucleic acid probe that hybridizes to the region of interest and a fluorophore. Melted wax is then added to the sample in a sample processing tube, which floats on top of the sample. The nucleic acid molecules in the sample are amplified, for example by adding polymerase to the sample (which may or may not already have wax on top of the sample). Amplification can occur, for example, under isothermal conditions. Following amplification, fluorescence of the sample is measured at a plurality of temperatures. For example, in some embodiments the sample is heated to a predetermined temperature and cooled while simultaneously measuring fluorescence of the sample. In some embodiments, the sample is heated to a predetermined temperature while simultaneously measuring fluorescence of the sample. The wax in the sample processing tube is solidified, and the sample processing tube containing the sample and the solidified wax is then discarded.

As used herein, the singular forms “a,” “an,” and “the” include the plural reference unless the context clearly dictates otherwise.

Reference to “about” a value or parameter herein includes (and describes) variations that are directed to that value or parameter per se. For example, description referring to “about X” includes description of “X”.

It is understood that aspects and variations of the invention described herein include “consisting” and/or “consisting essentially of” aspects and variations.

Where a range of values is provided, it is to be understood that each intervening value between the upper and lower limit of that range, and any other stated or intervening value in that stated range, is encompassed within the scope of the present disclosure. Where the stated range includes upper or lower limits, ranges excluding either of those included limits are also included in the present disclosure.

It is to be understood that one, some or all of the properties of the various embodiments described herein may be combined to form other embodiments of the present invention. The section headings used herein are for organizational purposes only and are not to be construed as limiting the subject matter described.

The nucleic acid to be isolated and/or analyzed can be DNA or RNA. In some embodiments, the DNA is genomic DNA, germline DNA, or cell-free DNA. (such as cell-free genomic DNA, cell-free fetal DNA, or cell-free tumor DNA).

In some embodiments, the fluorophores are included in a donor-quencher pair. In some embodiments, the fluorophores are attached to the nucleic acid probe. For example, a donor fluorophore can be attached to a first end of the nucleic acid probe and a quencher fluorophore can be attached to the second end of the nucleic acid probe. In some embodiments, the nucleic acid probe includes hybridization regions proximal to the fluorophores and a targeting region separating the hybridization regions. When the nucleic acid probe is not bound to the region of interest in the nucleic acid molecules form the sample, the hybridization regions can hybridize at the isothermal amplification temperature, which causes the donor fluorophore and the quencher fluorophore to be adjacent to each other. This configuration limits fluorescence detected by the fluorometer. However, if the targeting region of the nucleic acid probe binds the region interest of the nucleic acid molecule from the sample (e.g., hybridizes to the region of interest under isothermal. amplification conditions), the donor and quencher fluorophores are separated and fluorescence can be detected. Therefore, a large number of copies of the region of interest results in increased fluorescence, as a larger number of probes are bound to the region of interest.

Exemplary fluorophores include, but are not limited to 6-carboxyfluorescein; 5-carboxyfluorescein (5-FAM); boron dipyrromethene difluoride (BODIPY); N,N,N′,N′-tetramethyl-6-carboxyrhodamine (TAMRA); acridine, stilbene, 6-carboxyfluorescein (HEX), TET (Tetramethyl fluorescein), 6-carboxy-X-rhodamine (ROX), Texas Red, 2′,7′-dimethoxy-4′,5′-dichloro-6-carboxyfluorescein (JOE), SYBER Green, Cy3, Cy5, VIC® (Applied Biosystems), LC Red 640, LC Red 705, Yakima yellow, as well as derivatives thereof.

### Automated System

The automated system for isolating and analyzing nucleic acid samples includes a robotic pipettor, a robotic arm configured to transport one or more sample processing tubes, a nucleic acid isolation system, and a nucleic acid amplification and detection system that includes a fluorometer. In some embodiments, the system further includes one or more shakers, one or more heated incubators, a heated vessel comprising melted wax, a sample processing tube holder, a reagent rack, a liquid waste management system, a solid waste management system, one or more cooling racks, and/or a housing surrounding the system.

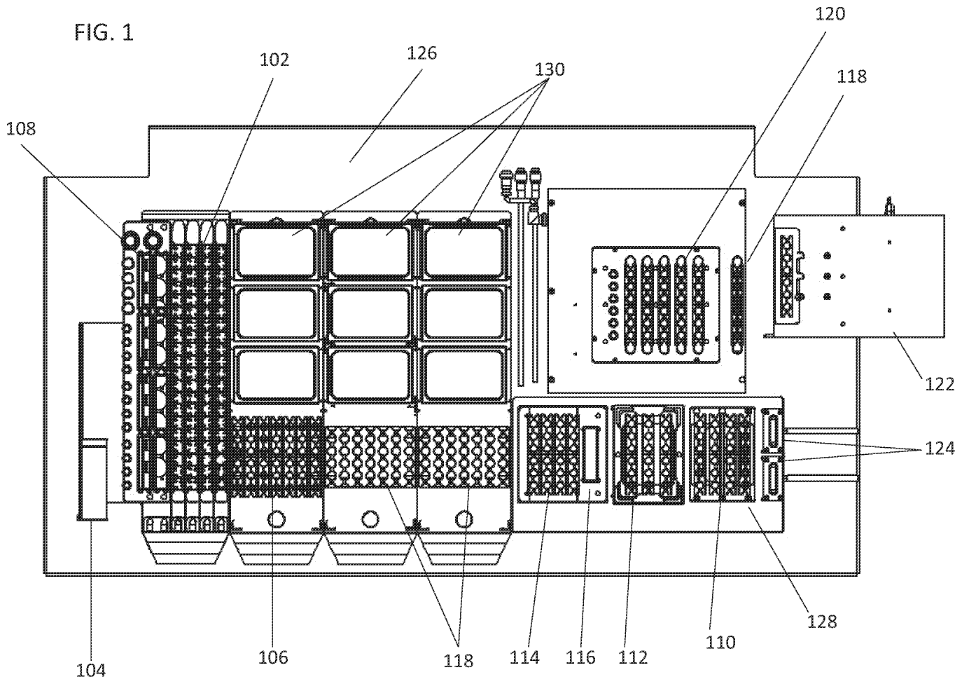

FIG. 1 illustrates an overhead schematic of an exemplary automated system. The robotic pipettor and the robotic arm configured to transport sample processing tubes is removed from this figure for easier viewing. The illustrated automated system includes a sample source tube holder 102, which is configured to hold a plurality of sample source tubes. The sample source tubes can be manually (and optionally randomly) inserted into individual slots within the sample source tube holder 102, or a rack configured to hold a number of sample source tubes can be inserted into the sample source tube holder 102. The sample source tubes holder contains biological samples (e.g., blood, plasma, saliva, solid tissue, semen, sputum, urine, etc.) from a subject. Optionally, the automated system includes a sample identification scanner 104, such as a barcode reader or radio-frequency identification (RFID) reader. The sample source tubes can include a sample identifier, such as a barcode or RFID tag, which is associated with a particular sample. Scanning of the sample allows the computer system to track the location and/or workflow (i.e., which processing and/or analysis steps have been completed). The automated system also includes a sample processing tube holder 106, which is configured to receive a plurality of connected sample processing tubes. In some embodiments, the plurality of connected sample processing tubes includes a sample processing tube identifier, such as a barcode or RFID tag, which can be scanned by the sample identification scanner 104 or a different sample identification scanner. A robotic pipettor comprising one or more pipettes can withdraw a sample from the sample source tube and dispense the sample in a sample processing tube. If the sample source tube includes a sample identifier which is scanned by the sample identification scanner 104, the computer system can track movement of the sample from the sample source tube to the sample processing tube. The automated system illustrated in FIG. 1 includes a reagent rack 108. The reagent rack 108 is configured to hold one or more reagents. In some embodiments, the reagent rack 108 is configured to receive on or more vessels that contain the regent, and in some embodiments the reagent rack 108 directly contains the one or more reagents. The reagents in the reagent rack are accessible by the robotic pipettor, which can move horizontally to position one or more pipettes over the desired reagent, lower the pipette vertically to dip a pipette tip into the reagent, and withdraw the desired amount of the reagent solution. The robotic pipettor can then move horizontally and/or vertically to dispense the reagent solution into a sample processing tube.

The automated system in FIG. 1 further illustrates a nucleic acid isolation system 110. The nucleic acid isolation system 110 includes a sample processing tube holder and a magnet. The robotic arm of the automated system is operable to transport the plurality of connected sample processing tubes to the sample processing tube holder of the nucleic acid isolation system 110. The robotic pipettor can dispense magnetically-responsive particles that bind to nucleic acids in the sample into the sample processing tube (for example, when the sample processing tube is located in the first sample processing tube holder 106 or the sample processing tube holder of the nucleic acid system 110). The magnet can move the magnetically-responsive particles to the inside wall surface of the sample processing tube while the robotic pipettor can dispense washing buffer to wash the particles and purify the nucleic acids bound on the particles.

The automated system illustrated in FIG. 1 further includes a shaker 112. The shaker 112 can include a sample processing tube holder configured to receive and a plurality of connected sample processing tubes. The sample processing tube holder can retain the plurality of connected sample processing tubes when the shaker shakes the sample processing tube holder (and thus the sample processing tubes held by the holder and the sample contained therein). Additionally, the automated system includes an incubator 114, which is configured to receive the plurality of connected sample processing tubes and heat samples contained within the sample processing tubes. The incubator 114 can be operated at a fixed temperature or can be operated to ramp up to a predetermined temperature. A heated vessel 116 is also included in the automated system, which can contain a wax heated to a temperature above the melting temperature of the wax. The robotic pipettor can be operated to withdraw melted wax from the heated vessel and dispense the melted wax in a sample processing tube, In some embodiments, the automated system includes one or more sample processing tube cooling racks 118. The sample processing tube cooling racks are sample processing tube holders, which are preferably held at or below room temperature. The plurality of connected sample processing tubes can be transported to one of the cooling racks 118 by the robotic arm, for example after the plurality of connected sample processing tubes was heated in the heated incubator. In some embodiments, a sample processing tube containing melted wax is transported to the sample processing tube cooling rack 118 to allow the melted wax to solidify. The solidified wax can be used to seal the tube and prevent the sample solution from spilling out of the tube. If the automated system includes a plurality of sample processing tube cooling racks 118, as illustrated in FIG. 1, the one or more sample processing cooling racks 118 can be positioned adjacent to each other or in different locations throughout the sample processing system. For example, in the embodiment illustrated in FIG. 1, three sample processing cooling racks 118 are identified, with two adjacent to each other and positioned between the sample processing tube holder 106 (which, if unheated can be considered a sample processing cooling rack, as it is similarly configured to receive a plurality of connected sample processing tubes) and the incubator 114. A third sample processing cooling rack 118 in in the embodiment illustrated in FIG. 1 is positioned between a fluorometer 120 and a solid waste management system 122. The fluorometer 120 includes a heater, a light source and an optical detector disposed below a sample processing tube holder. The plurality of connected sample processing tubes can be transported to the sample processing tube holder of the fluorometer 120 by the robotic arm. The sample processing tube holder of the fluorometer 120 is heated or configured to be heated, either at a fixed temperature or at a variable temperature (i.e., the temperature can be ramped up or down). The sample processing tube holder of the fluorometer 120 has a clear or open bottom, which allows the light source and the optical detector to detect fluorescence of the sample in the sample processing tube.

The automated system illustrated in FIG. 1 also includes a solid waste management system 122 and a liquid waste management system 124. The solid waste management system 122 is configured to receive solid waste, such as used pipette tips and/or used sample processing tubes. In some embodiments, melted wax is dispensed in the sample processing tube, which solidifies at room temperature. The wax floats to the top of the sample contained within the sample processing tube, and once solidified, traps the liquid in the sample processing tubes such that it does not leak. Thus, even though liquids can be contained within the sample processing tube, the sample processing tube can be disposed of in the solid waste management system 122. The liquid waste management system 124 includes one or more liquid waste ports and a liquid waste conduit. The liquid waste port is fluidly connected to the liquid waste conduit, which can be fluidly connected to a waste collection vessel or a sewage line. The robotic pipettor can dispense spent reagents in the liquid waste port, and the liquid waste and flow through the liquid waste conduit for disposal.

The automated system can also include one or more pipette tip containers 130. The pipette tip containers contain a plurality of pipette tips, which can be attached to the robotic pipettor. To limit cross contamination, the robotic pipettor is configured to receive a new pipette tip after dispensing a liquid and before contacting a new liquid. For example, the robotic pipettor configured with a pipette tip can withdraw a sample from a sample source tube and dispense the sample in a sample processing tube. Prior to adding a reagent to the sample processing tube, the robotic pipettor will dispose of the first pipette tip in the solid waste management system, move above a pipette tip contained by the one or more pipette tip containers 130, and lower the robotic pipettor to the pipette tip thereby securing the pipette tip to the robotic pipettor. Once the new pipette tip is attached to the robotic pipettor, the robotic pipettor can withdraw the reagent (for example from the reagent rack 108) and dispense the reagent in the desired sample processing tube.

Components of the automated sample processing and analysis system can be attached to a surface 126. For example, in some embodiments, one or more of the sample source tube holder 102, the sample identification scanner 104, a sample processing tube holder 106, the reagent rack 108, the nucleic acid isolation system 110, the a shaker 112, the incubator 114, the heated vessel 116 configured to contain the melted wax, the one or more sample processing tube cooling racks 118, and/or the fluorometer 120 is attached to the surface 126 of the system. In some embodiments, one or more components of the solid waste management system 122 or the liquid waste management system 124 (such as the liquid waste port) is attached to the surface of the system 126. Components of the system can be attached directly to the surface 126, can be mediated by a module mounting plate 128. The module mounting plate 128 is configured to attach to the surface and receive one or more components of the system. For example, in the embodiment illustrated in FIG. 1, the module mounting plate 128 is configured to receive the incubator 114, the shaker 112, the nucleic acid isolation module 110, and two liquid waste ports of the liquid waste management system 124.

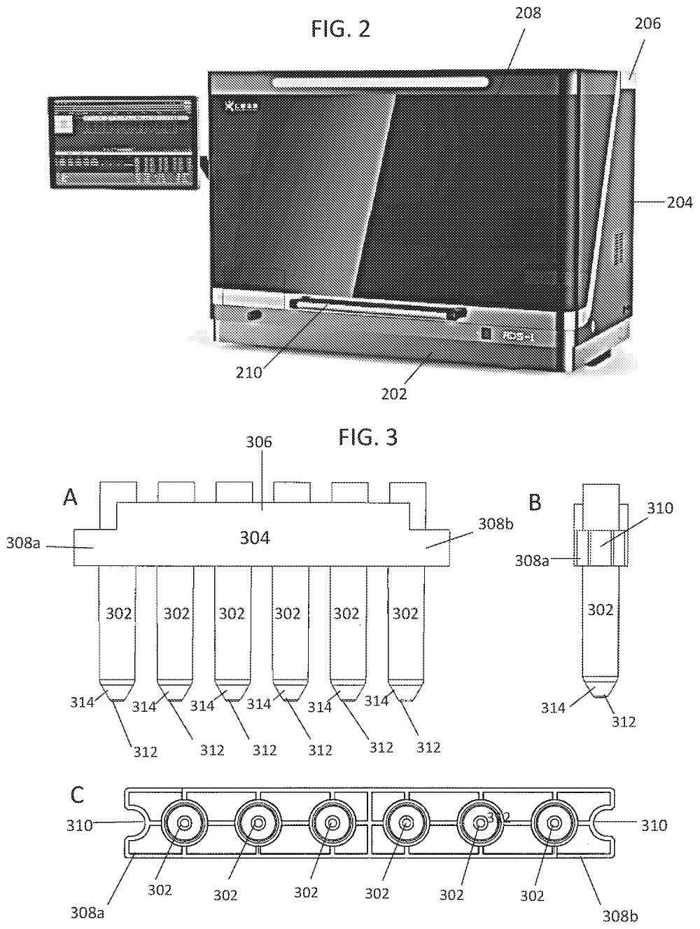

The components of the system can be enclosed in a housing, as illustrated in FIG. 2. The housing can have a base 202, sidewalls 204, a roof 206, and a back (not illustrated). The housing can also include a lid 208, which can be opened to expose the components of the system, for example to refill reagents, sample processing tubes, or pipette tips; to add or remove sample source tubes, and/or empty the solid waste management system. Optionally, a handle 210 can be included on the lid 208 to ease opening or closing of the lid 208.

In some embodiments, the housing includes a ventilation system. The ventilation system can include an air filter and an air pump, and can be configured to provide filtered air to the system enclosed by the housing. The ventilation system can also withdraw air from the enclosed system to provide a continuous negative pressure to the system. The filtered air provided to the system helps limit cross contamination of the samples, while the negative pressure in the system limits escape of evaporated reagents outside of the system.

One or more ultraviolet (UV) lights can optionally be included in the system. The UV light can sterilize the system, for example when the system is not processing or analyzing samples. The UV light can be positioned on the inside of the housing, for example on the sidewall or the lid of the housing, and can be configured to emit UV on the components of the system. In some embodiments, the system includes a UV light not attached to the housing; for example, a UV light can be attached to the solid waste management system and can be configured to emit UV light to sterilized solid waste contained within the waste management system.

The automated system includes one or more robotic arms configured to transport the plurality of connected sample processing tubes to various components throughout the system. The robotic arm can include an engagement region, which is configured to engage the plurality of connected sample processing tubes such that the plurality of connected sample processing tubes can be transported. For example, the engagement region of the robotic arm can be lowered to engage the plurality of sample processing tubes, and can be raised after engaging the plurality of sample processing tube to raise the plurality of sample processing tubes. By way of example, the engagement region can include a hook, a clamp, a magnet, a vacuum, or any other device to temporarily secure the plurality of sample processing tube to the engagement region of the robotic arm. The plurality of connected sample processing tubes can include a ridge, notch, cutout, handle, or any other feature that facilitates engagement with the robotic arm. The robotic arm is configured to be movable in a vertical direction to raise and/or lower the plurality of connected sample processing tubes. The robotic arm is also configured to move in a horizontal plane to transport the sample processing tubes within the system.

In some embodiments, the plurality of connected sample processing tubes is a multi-well plate. Each well in the multi-well plate is a separate sample processing tube that can receive a sample. The multi-well plate has a translucent or transparent bottom, which allows the fluorometer to detect fluorescence of the sample from below. In some embodiments, the plurality of connected sample processing tubs is a plurality of linearly arrange sample processing tubes. In some embodiments, the plurality of sample processing tubes includes two or more, three or more, four or more, five or more, or six or more sample processing tubes. In some embodiments, the plurality of sample processing tubes includes 24 or fewer, 20 or fewer, 16 or fewer, 12 or fewer, 8 or fewer, or 6 or fewer sample processing tubes. In some embodiments, the sample processing tubes have a volume of about 100 microliters (μL) or more (such as about 250 μL or more, about 500 μL or more, about 1 mL or more, about 1.5 mL or more, or about 2 mL or more). In some embodiments, the sample processing tubes have a volume of about 10 mL or less (such as about 5 mL or less, about 4 mL or less, about 2 mL, or less, about 1.5 mL or less, about 1 mL, or less, about 500 μL or less, or about 250 μμL or less). To limit spillage or cross-contamination, the maximum volume of liquid in the sample processing tube is preferably substantially less than the volume of the sample processing tube. For example, in some embodiments, the maximum volume of liquid in the sample processing tube at any time during sample preparation or analysis about 50% or less (such as about 40% or less, about 30% or less, about 20% or less, or about 10% or less) of the maximum volume of the sample processing tube. FIG. 3A-C illustrates a strip comprising six linearly arranged connected sample processing tubes. FIG. 3A shows a front view of the strip of linearly arranged connected sample processing tubes. The sample processing tubes 302 are connected by a connector 304. In the illustrated example, the connector 304 includes a mid-region 306 and side regions 308a and 308b. The engagement region of the robotic arm can engage the side regions 308a and 308b. FIG. 3B illustrates a side-view of the strip of linearly arranged connected plurality of sample processing tubes 302. Although the side-view of the strip illustrated in FIG. 3B shows a single side, the opposite side of the strip is similarly designed. The side region 308a includes a rounded cutout 310 through the vertical dimension of the side region 308a. The engagement region of the robotic arm can engage the cutout 310 to temporarily attach the strip to the robotic arm. The sample processing tubes 302 in in the strip include a flat and transparent (or translucent) bottom 312, which limits error of fluorescence measured by the fluorometer. The lower portion 314 of the sample processing tubes 302 can be tapered to consolidate the liquid toward the bottom of the tube. FIG. 3C shows a top view of the strip of linearly arranged connected sample processing tubes 302. The sample processing tubes 302 are connected by a connector 304, which includes side regions 308a and 308b and mid-region 306. The mid-region 306 includes a plurality of cross braces, which enhance stability of the strip. As shown in the top view, both side regions 308a and 308b include a cutout 310.

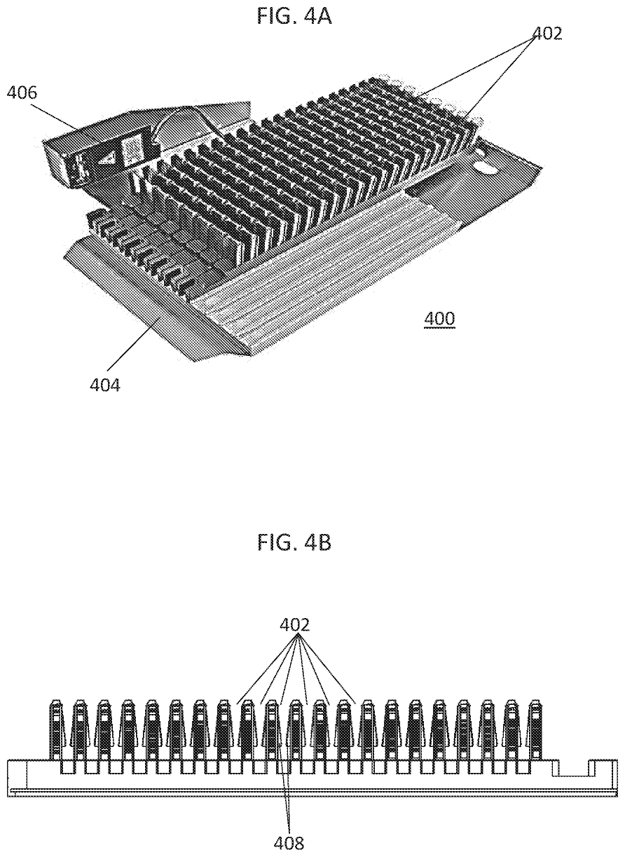

Samples contained in sample source tubes are positioned in the sample source tube holder. Positioning of the sample source tubes in the sample source tube holder can be performed manually (i.e., by a technician) or by an automated robot. The sample source tube holder includes a plurality of slots configured to receive a single sample source tube. In some embodiments, the sample source tube holder comprises 4 or more, 8 or more, 12 or more, 16 or more, 20 or more, 40 or more, 60 or more, 80 or more, 100 or more, 120 or more, 140 or more, or 160 or more slots that are configured to receive a sample source tube. The slots can be configured in any suitable arrangement, such as in a plurality of rows and columns. A sample identification scanner (such as a barcode scanner or RFID scanner) is positioned adjacent to or attached to the sample source tube holder. In some embodiments, the sample identification scanner and/or the slots of the sample source tube holder are movable to position the sample source tube such that the sample identifier on the sample source tube can be scanned by the sample identification scanner. If RFID tags are used for the sample identifier, it is preferable that the scanned sample source tube is positioned sufficiently distant from other sample source tubes to avoid incorrectly identifying a sample source tube. Scanning of a particular sample source tube associates a sample with a location within the system, and the sample location can be communicated to the computer system. As the sample is processed and analyzed throughout the system, the location of the sample can be tracked. Therefore, analysis results such as fluorescence readings and/or generated melting curves) can be associated to the sample. FIG. 4A illustrates an exemplary sample source tube holder 400. The sample source tube holder 400 includes a plurality of slots 402 configured in 20 columns and 8 rows. Each slot 402 is configured to receive a sample source tube. In some embodiments, the sample source tube holder 402 include a module mounting plate 404, which can be used to attach the sample source tube holder 400 to a surface of the system. Attached to the sample source tube holder 402 is a barcode scanner 406. The barcode scanner 406 is configured to scan sample source tubes held in the slots 402. FIG. 4B shows a side view of one row of slots 402 of the sample source tube holder 400. Optionally, each slot 402 includes a tube clamp 408 configured to secure the sample source tube in the slot. The tube clamp 408 can include, for example, a pair of springs attached to the inner walls of the slot 402. When a sample source tube is positioned in the slot 402, the springs are compressed against the inner wall and exert a pressure on the sample source tube to secure the sample source tube.

The robotic pipettor can withdraw some or all of the sample contained within the sample source tube and dispense the sample in a sample processing tube. The sample processing tubes are held by a sample processing tube holder. Preferably, the sample processing tube holder is located near or adjacent to the sample source tube holder. This configuration minimizes the distance traveled by the robotic pipettor during transfer of the sample from the sample source tube to the sample processing tube, and can reduce risk of drip from the pipette to the surface of the system, a system component, or a separate sample processing tube. The sample processing tube holder is configured to hold a plurality of sample processing tubes, which may be connected. In some embodiments, the sample processing tube holder includes a plurality of holes in a platform. The bottom portion of the sample processing tubes can fit through the holes in the platform of the sample processing tube holder. In some embodiments, the plurality of sample processing tubes are linearly connected in a strip, and the sample processing tube holder includes cutouts on the edges of the platform through which the bottom portion of the sample processing tubes on the ends of the strip can fit through. In this configuration, the robotic arm can engage the side regions of the strip to lift the plurality of sample processing tubes from the sample processing tube holder. In some embodiments, the platform of the sample processing tube holder includes a groove, which can receive a portion of the connector connecting the sample processing tubes. FIG. 5 illustrates an exemplary sample processing tube holder 500 configured to seven strips of linearly connected sample processing tubes, each strip comprising six linearly arranged, connected sample processing tubes. The sample processing tube holder includes a platform 502 elevated by supports 504. In the illustrated example, the platform includes seven rows of four linearly arranged holes 506. The strip includes six sample processing tubes, and the bottom portions of the inner four sample processing tubes fit in the four holes 506. At both edges of the platform 502, the sample processing tube holder includes cutout 508 and cutout 510. Cutout 508 and cutout 510 are disposed linearly with respect to the four holes 506. Side regions of the strip held by the sample processing tube holder can protrude from the platform unobstructed, and the robotic arm can engage the side regions of the strip. The bottom portion of the two sample processing tubes at the ends of the strip fit within cutout 508 and cutout 510 A groove 512 connects the holes 506, cutout 508, and cutout 510. The groove 51 can receive a connector that connects the plurality of sample processing tubes in the strip, and can stabilize the strip to minimize movement.

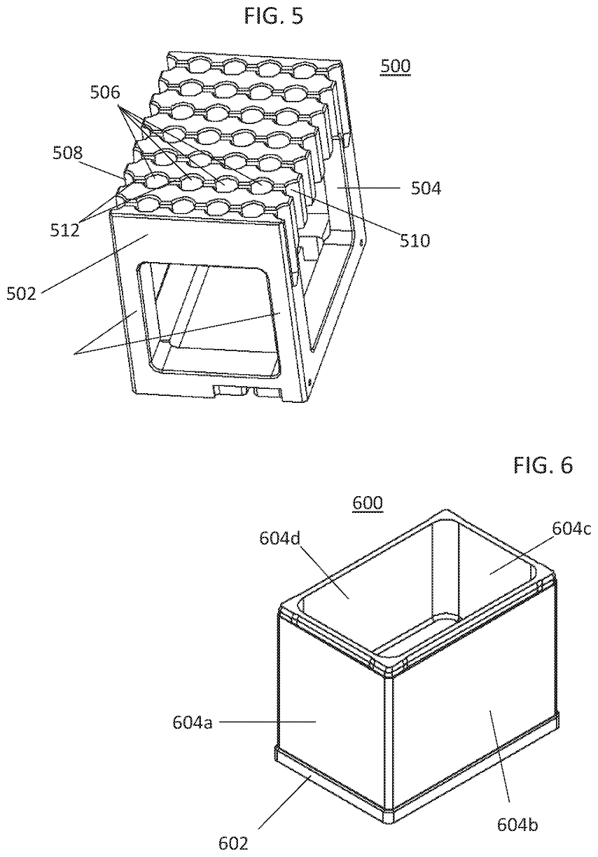

The robotic pipettor can receive a new pipette tip to avoid cross contamination of samples and/or reagents. Unused pipette tips can be held in one or more pipette tip containers. The pipette tip containers are configured to receive a cartridge comprising a plurality of pipette tips. The pipette tips include a liquid contact end and a pipette contact end. The liquid contact end of the pipette tip is generally tapered, and includes an opening to allow reagents or samples to flow through into the pipette. The pipette contact end also includes an opening that can engage the pipette through a friction fit, which secures the inner wall of the pipette contact end of the pipette to the outer wall of the lower portion of the pipette. Thus, the diameter of the pipette tip is slightly larger than the diameter of the pipette. A cartridge of pipette tips can be placed into a pipette tip container. FIG. 6 illustrates an exemplary pipette tip holder 600. The pipette tip container 600 includes a base 602 and sidewalls (604a, 604b, 604c, and 604d). The top of the pipette tip container is open, which allows a cartridge containing pipette tips to be loaded into the pipette tip container 600. The opening also allows pipettes of the robotic pipettor access the pipette tips. The distances between the sidewalls 604a-d of the pipette tip container 600 are configured to receive the cartridge of pipette tips and to limit movement of the cartridge once loaded into the pipette tip container 600. The base 602 of the pipette tip container 602 can be attached to the surface of the system.

Reagents held by the reagent rack can include any desirable reagent for the sample being processed or the method of analysis. In some embodiments, the reagents include, for example, a wash buffer, magnetically responsive particles (which can be suspended in a liquid, such as wash buffer, saline, etc.), a lysis buffer, deionized. water, nucleic acid probes (such as a fluorescently labeled nucleic acid probe, such as a molecular beacon), one or more fluorophores, controls (e.g., positive controls or negative controls), and/or enzymes (such as lysis enzymes or amplification enzymes).

Used pipette tips are disposed of in a solid waste management system. The solid waste management system includes a solid waste port and a waste chute. The solid waste port is configured to receive sample processing tubes and pipette tips after use. In some embodiments, the solid waste port comprises one or more slots for disconnecting the used pipette tips from the robotic pipettor. The one or more slots are sized to the diameter of the pipettes such that the distance between the edges of the slots is larger than the diameter of the pipette but smaller than the diameter of the pipette tip. The robotic pipettor can slide the pipette into the slot horizontally, with the pipette tip positioned underneath the slot. The robotic pipettor can then raise the pipette, causing the top of the pipette to catch on the underside of the slot. Once the pipette tip catches on the underside of the slot and the pipette continues to be raised, the pipette tip dissociates from the pipette and falls into the chute. The chute of the solid waste management system is connected to a container, which can receive the solid waste. In some embodiments, the container is configured to a sensor, which is connected to a computer system. When the sensor detects the amount of waste in the solid waste system is above a predetermined threshold or full, an indicator (such as an audible or light alarm) can be triggered to alert a user or to suspend operation of the system. in some embodiments, the solid waste management system includes a UV light to sterilize the solid waste. The UV light can be positioned, for example, to shine onto the waste collected in the container.

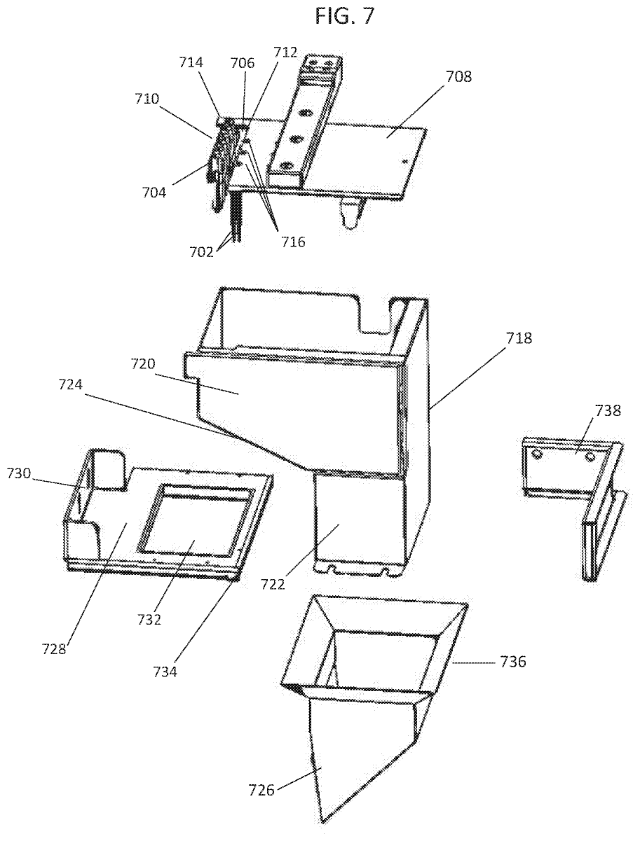

FIG. 7 illustrates an exemplary solid waste management system, in an exploded view. Tips 702 and a strip of sample processing tubes 704 are illustrated in the solid waste port 706 to show how the solid waste management system receives the solid waste. The solid waste port 706 is disposed on the lid 708 of the solid waste management system. The solid waste port 706 includes an open side 710 and an edged side 712. The open side 710 of the solid waste port 706 is proximal to the other components of the system. The solid waste port 706 can include an arm 714 on either side of the solid waste port 706 to define the opening of the port 706. For clarity, only a single arm 714 is shown, but a similar arm can be disposed on the opposite side of the solid waste port 706. To dispose of sample processing tubes, the robotic arm can move the sample processing tubes to the solid waste port 706, and release the sample processing tube such that the sample processing tube falls to the lower regions of the solid waste management system. In some embodiments, the sample processing tubes are moved into the solid waste port 706 in a horizontal motion such that the sample processing tubes are positioned below the solid waste port 706 upon release by the robotic arm. In some embodiments, the sample processing tubes are released from above the solid waste port 706 such that they fall through the solid waste port 706 at upon release by the robotic arm. One or more slots 716 are disposed along the edged side 712 of the solid waste port 706. The robotic pipettor can move the one or more pipettes with a pipette tip 702 attached into the solid waste port (either horizontally through the open side 710 or from above), and into the one or more slots 716 horizontally. Once the one or more pipettes are positioned in the one or more slots 716, the robotic pipettor can move the pipettes upward, causing the one or more pipette tips 702 to catch on the underside of the one or more slots 716, dissociate from the one or more pipettes, and fall into the lower regions of the solid waste management system. The lid 708 fits on top of a chute 718, which includes an upper portion 720 and a lower portion 722. The upper portion 720 of the chute 718 has a larger horizontal cross section than the lower portion 722. A sloped surface 724 along the bottom of the upper portion 720 funnels solid waste deposited into the solid waste port 706 into the lower portion 722 of the chute 718. The solid waste flows through the lower portion 722 of the chute 710 and into a waste container (not show). In some embodiments, the waste management system includes an adapter 726, which connects the chute 718 to the container. The adapter 726 can be included to ensure a tight fit between the container and the chute 718, which can limit spillage of solid waste. In some embodiments, the solid waste management system includes an attachment plate 728. The attachment plate 728 can secure the chute 718 and/or adapter 726 to the side of the system. For example, the attachment plate 728 can include an attachment region 730, through which bolts or any other suitable faster can attach the attachment plate 728 to the side of the system. The lower portion 722 of the chute 718 can attach to the top of the attachment plate 728 such that the opening of the lower portion 722 of the chute 718 is disposed above an opening 732 in the attachment plate 728. The adapter 726 can attach to the lower portion of the attachment plate 728. In some embodiments, the adapter 726 is removable from the attachment plate 728, and may be removed, for example, to remove any clogs in the chute 718 or adapter 726. In some embodiments, the attachment plate 728 includes a slot 734 on the underside of the attachment plate 728, and the adapter 726 includes a lip 736 on the top of the adapter 726. The lip 736 of the adapter 726 can slide into the slot 734 to attach the adapter 726 to the attachment plate 728. Optionally, the waste management system also includes a fastener 738, which can secure the lower portion 722 of the chute 718 to the side of the system.

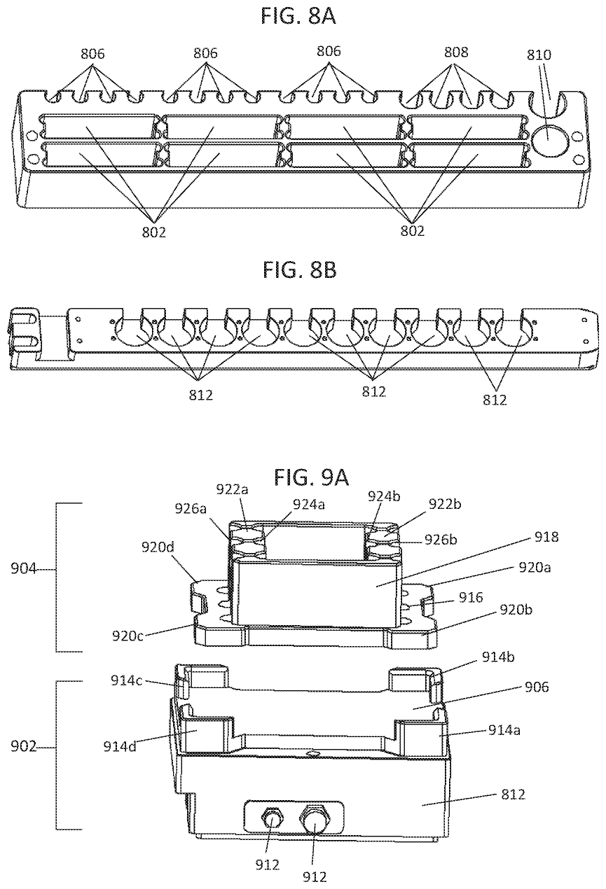

The automated system can also include a reagent rack configured to hold one or more reagents. The reagent rack can include one or more troughs and/or one or more reagent bottle holders, In some embodiments, the reagent rack includes two or more troughs of different sizes and/or two or more reagent bottle holders of different sizes. The troughs can contain reagents for which a relatively large volume of liquid is used during the sample processing or analysis, such as wash buffers. In some embodiments, the troughs have a maximum volume of about 10 mL or more (such as about 25 mL or more, about 50 mL or more, about 100 mL or more, about 250 mL or more, or about 500 mL or more) of reagent. In some embodiments, the troughs have a maximum volume of about 1 L or less (such as about 500 mL or less, about 250 mL or less, about 100 mL or less, about 50 mL or less, or about 25 mL or less). In some embodiments, the reagent rack includes 1, 2, 3, 4, 5, 6, 7, 8 or more troughs. In some embodiments, the troughs contain the liquid directly. In some embodiments the troughs contains a liner or secondary container, which contains the reagent In some embodiments, the reagent bottle holders receive a bottle, which contains a reagent. In some embodiments, the bottles have a maximum volume about 0.5 mL or more, about 1 mL or more, about 2 mL or more, about 5 mL or more, about 10 mL or more, about 15 mL or more, or about 25 mL or more. In some embodiments, the bottles have a maximum volume about 50 mL or less, such as about 25 mL or less, about 10 mL or less, about 5 mL or less, about 2 mL or less, or about 1 mL or less. In some embodiments, the reagent rack includes 1 or more reagent bottle holders (such as 2 or more, 4 or more, 8 or more, 12 or more, 16 or more, 20 or more, 24 or more, or 28 or more reagent bottle holders). In some embodiments, the reagent rack includes reagent bottle holders of different sizes, such as two or more, three or more, or four or more different sizes. Reagent or reagent bottles can be manually placed in the reagent rack. During operation, the robotic pipettor is operated to lower a pipette tip into a reagent trough or reagent bottle held by the reagent rack and withdraw the desired amount of reagent into the pipette tip. FIGS. 8A and 8B illustrate exemplary reagent racks. The reagent rack illustrated in FIG. 8A includes eight reagent troughs 802, arranged in two rows of four troughs. Along the edge of the reagent trough, there is a plurality of opening, which can receive reagent bottles. The openings are of three different sizes: small openings 804, medium openings 806, and large openings 808, which can receive different size reagent bottles. The reagent rack shown in FIG. 8B includes a plurality of linearly arranged openings 810 of a single size.

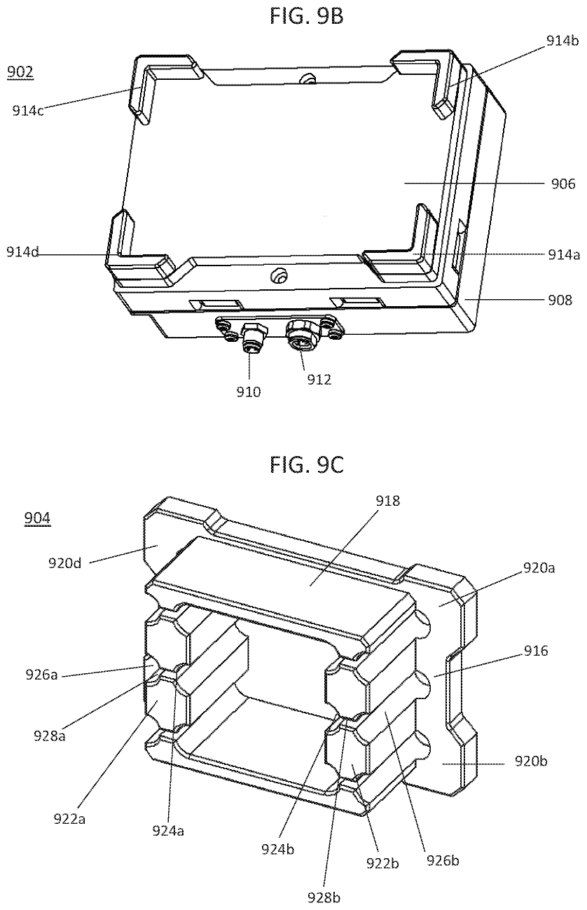

The automated system can include a shaker. The shaker can be operated to mix a sample in a sample processing tube at a desired speed. In some embodiments, the shaker is operated to vortex the sample in the sample processing tube. In some embodiments, the shaker is operated to rock the sample in the sample processing tube. A sample processing tube holder can be placed on or attached to the top of the shaker (i.e., on a platform of the shaker), and can hold the sample processing tube during shaking. In some embodiments, such as when a plate is used for the sample processing tubes, the plate can be placed directly on the platform of the shaker. The platform of the shaker can include a raised lip or corners, which can prevent the sample processing tube holder or plate form sliding off the platform of the shaker during operation of the shaker. FIG. 9A illustrates an exemplary shaker 902 with a sample processing tube holder 904 (shown above instead of on top of the shaker for clarity). A tilted view of the shaker 902 is shown in FIG. 9B, and a tilted view of the sample processing tube holder 904 is shown in FIG. 9C. The shaker 902 includes a shaking platform 906 attached to a base 908. The base 908 includes a motor and electronic circuitry to operate and shake the platform 906 at a suitable speed. The base also includes a power port 910, which can be connected to a power source to provide power to the shaker, and a communication port 912, which can be connected to the computer system to operate the shaker 902, The computer system can control, for example, when the shaker 902 is turned off or on, or the rate of shaking of the shaker 902. The platform 906 can include raised corners 914a, 914b, 914c, and 914d. In some embodiments, the platform includes a raised lip (not shown) around the perimeter of the platform 906. The raised corners or lip are sized to receive the sample processing tube holder 904, and can secure the sample processing tube holder to the platform 906 such that the sample processing tube holder 904 does not slide off the sample processing holder 904 during operation of the shaker 902. The sample processing tube holder 904 includes a bottom segment 916 and a raised segment 918. The bottom segment is sized to fit on the platform 906 of the shaker 902. The corners 920a, 920b, 920c and 920d of the bottom segment 916 engaged the raised lip or corners 914a, 914b, 914c, and 914d of the platform 906. The raised segment 918 of the sample processing tube holder 904 includes two opposite sidewalls 922a and 922b, each with cutouts to receive the bottom portions of linearly connected sample processing tubes. The sidewalls 922a and 922b can include one or more inner cutouts 924a and 924b and one or more outer cutouts 926a and 926b. The sample processing tube on the ends of a strip of a plurality of linearly arranged, connected sample processing tube can engage the outer cutouts 926a and 926b, and the first inner sample processing tubes in the strip can engage the inner cutouts 924a and 924b. The sidewall 922a can also include a groove 928a connecting the inner cutout 924a with the outer cutout 926a, and the sidewall 922b can also include a groove 928b connecting the inner cutout 924b with the outer cutout 926b. The grooves 928a and 928b can receive the connector of the plurality of sample processing tubes.

The automated system can include one or more heated incubators configured to receive the one or more sample processing tubes. The heated incubator is set to a predetermined temperature, such as about 30° C. or warmer, about 35° C. or warmer, about 40° C. or warmer, about 50° C. or warmer, about 60° C. or warmer, about 65° C. or warmer, about 70° C. or wanner, or about 80° C. or warmer, In some embodiments, the heated incubator is set to a temperature of about 100° C. or cooler, about 90° C. or cooler, about 80° C. or cooler, about 70° C. or cooler, or about 65° C. or cooler. The heated incubator includes a sample processing tube holder, which is configures so that the robotic arm can place the sample processing tubes in or remove the sample processing tubes from the heated incubator. The sample processing tube holder is disposed in the heated incubator, and includes openings on either side such that the robotic arm can engage with the end regions of a sample processing strip. In some embodiments, the heated incubator comprises a heated vessel, which can contain wax (such as paraffin). The heated vessel is heated to a temperature above the melting temperature of the wax. In some embodiments, the melting temperature of the wax is above room temperature (such as about 30° C. or higher, about 40° C. or higher, about 50° C. or higher, or about 60° C. or higher), In some embodiments, the melting temperature of the wax is about 70° C. or lower (such as about 65° C. or lower, about 60° C. or lower, about 55° C. or lower, or about 50° C. or lower), The heated vessel can be integrated with, or separate from, the heated incubator with the sample processing tube holder.

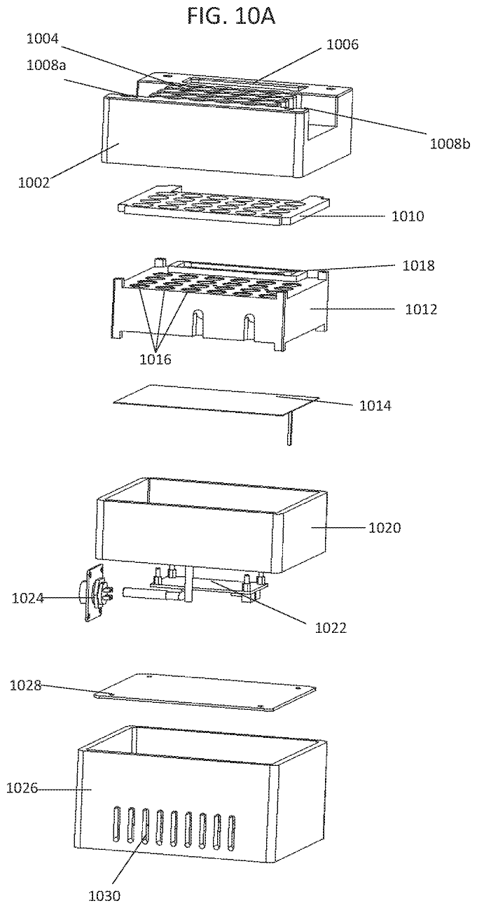

FIG. 10A illustrates an exploded view of an exemplary heated incubator with a sample processing tube holder and an integrated heated vessel configured to contain melted wax. The heated incubator includes a top layer 1002, which includes the sample processing tube holder 1004 and a vessel 1006. The vessel 1006 can include the wax, and is open at the top and sealed at the bottom. The sample processing tube holder 1004 includes a plurality of holes configured to receive the sample processing tubes. The ends 1008a and 1008b of the sample processing tube holder are open to provide space for the robotic arm to place or retrieve the sample processing tubes from the incubator. The conductive block 1012 fits into the underside of the top layer 1002, which is heated to a desired temperature by a heating element 1014. The heating element 1014 can be heated, which warms the conductive block 1012 to the desired temperature. The conductive block 1012 includes a plurality of holes that align with the holes in the top layer 1002. The bottom portions of sample processing tubes placed in the top layer 1002 sit within the holes 1016 of the conductive block 1012 so that the contents of the sample processing tubes are heated. The conductive block 1012 also includes a vessel portion 1018, which can receive and warm the vessel 1006 of the top layer 1002. The top layer 1002, the conductive block 1012, and the heating element 1014 assemble into an inner heated housing 1020, with the heating element 1014 forming the floor of the heated housing 1020. The heated housing 1020 and the heating element 1014 are operated by a control unit 1022, which is connected to a power and/or data port 1024. The power and/or data port can be connected to a power source, which can supply power to the heated incubator, and/or a computer system, which can control the temperature of the heated incubator. The top layer 1002, the conductive block 1012, and the heating element 1014 assembled into the heated housing 1020 can be disposed in an outer housing 1026, which is optionally insulated. The housing can also include a floor 1028. In some embodiments, the outer housing 1026 includes a plurality of vents 1030. FIG. 1013 illustrates a vertical cross-section of the heated incubator.

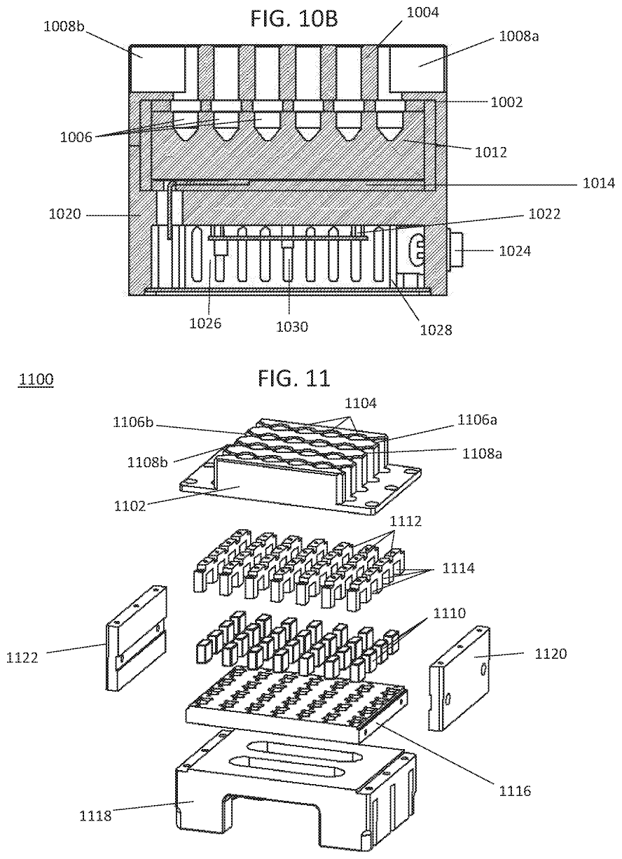

The nucleic acid isolation system of the automated sample processing and analysis system includes a sample processing tube holder and one or more magnets. Magnetically-responsive particles are dispensed into a sample processing tube and can bind to nucleic acid molecules in the sample contained within the sample processing tube. For example, the robotic pipettor can withdraw magnetically-responsive particles contained in a reagent bottle held by the reagent rack and dispense the magnetically-responsive particles into the sample processing tube. The magnet of the nucleic acid isolation system can interact with the magnetically-responsive particles that bind to the nucleic acids in the sample such that when the robotic pipettor withdraws the liquid from the sample processing tube the magnetically-responsive particles (and thus, the nucleic acid molecules that are bound to the magnetically-response particles) remain in the sample processing tube. When the magnet engages the magnetically-responsive particles in the sample processing tube, the magnet is considered to be in an active configuration. In some embodiments, the magnet is fixed, and thus in an active position when the sample processing tube containing magnetically-active particles is positioned in the sample processing tube holder of the nucleic acid isolation system. In some embodiments, the magnet is configured to be operated between an active and a passive configuration. The magnet can be operated, for example, by the computer system. The magnet can be switched between the active and passive configurations for example, by controlling electricity flowing through an electromagnet or by physically positioning the magnet in an active or a passive position. FIG. 11 illustrates an exploded view of a nucleic acid isolation system 1100. The nucleic acid isolation system 1100 includes a sample processing tube holder 1102. The sample processing tube holder 1102 includes a plurality of holes 1104 configured to receive the bottom portions of the sample processing tube. The sample processing tube holder also includes two opposite ends 1106a and 1106b, each with cutouts 1108a and 1108b to receive the bottom portions of the terminal sample processing tubes in a strip of linearly connected sample processing tubes. The nucleic acid isolation system 1100 further includes a plurality of magnets 1110, which are held in place by holding strips 1112. The holding strips 1112 comprise a plurality of notches 1114, which receive the magnets 1110 and hold the magnets in place (i.e., in an active configuration). The holding strips 1112 engage a magnet mounting plate 1116 to secure the magnets 1110 in a fixe position. The assembly including the magnet mounting plate 1116, holding strips 1112, and magnets 1110 is fastened to a bottom plate 1118, which attaches to the surface of the system (or to a module mounting plate on the surface of the system). The nucleic acid isolation system 1100 can also include sidewalls 1120 and 1122 to further secure the system.

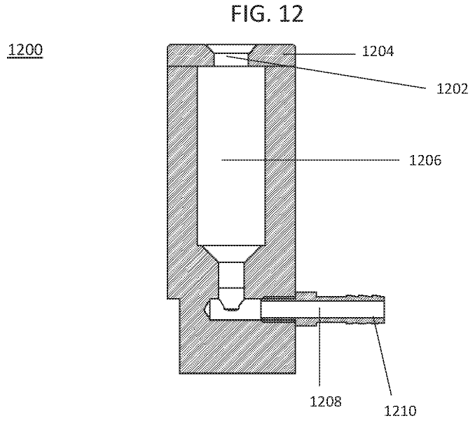

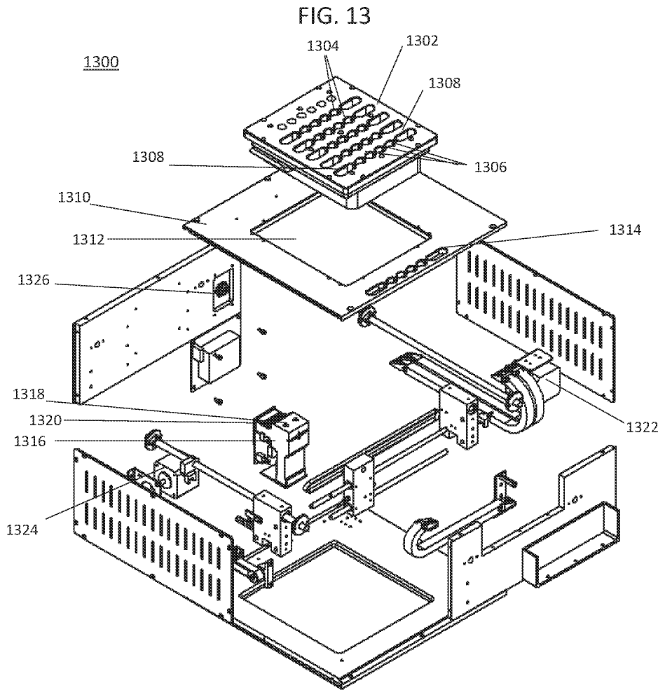

Liquid waste produced during sample preparation can be disposed of using a liquid waste management system, which can be included in the automated system. The liquid waste management system includes one or more liquid waste ports and a conduit configured to drain the liquid waste from the liquid waste port. The liquid waste conduit is fluidly connected to a liquid waste container or sewer system for treatment or disposal of the liquid waste. The robotic pipettor can withdraw liquid wasted (e.g., spent reagent or sample) from a sample processing tube and dispense the liquid waste into the liquid waste port. The liquid then drains from the automated system through the conduit. FIG. 12 illustrates an exemplary liquid waste port for use with a liquid waste management system. The liquid waste port 1200 includes a lid 1202 with a hole 1204 in the lid 1202 through which liquid waste can be dispensed. The hole 1204 opens into a chamber 1206, which can hold liquid waste until it drains. The chamber 1206 is fluidly connected to a conduit connector 1208 to which the conduit is attached. The conduit connector 1208 can include one or more barbs 1210 to secure the conduit onto the conduit connector.

Once the sample has been processed, it can be analyzed by the fluorometer. In some embodiments, the sample is amplified in the fluorometer, for example by isothermal amplification. The fluorometer includes a heating unit, which is operated to heat the sample processing tube rack in the fluorometer, thereby heating the samples contained within the sample processing tubes held by the sample processing tube rack to a desired temperature. In some embodiments, the fluorometer is set to or is ramped up to a predetermined isothermal amplification temperature above room temperature, such as between about 30° C. and about 80° C. (such as between about 30° C. and about 60° C., about 37° C. and about 47° C., or about 42° C.). For example, in some embodiments, the fluorescence is measured every 10 minutes or more frequently, every 5 minutes or more frequently, every 3 minutes or more frequently, every 2 minutes or more frequently, every minute or more frequently, or every 30 seconds or more frequently. In some embodiments, the fluorometer measures fluorescence of the sample during isothermal amplification. In some embodiments, isothermal amplification proceeds for about 20 minutes or more (such as about 30 minutes or more, or about 40 minutes or more). In some embodiments, a melting curve is generated by measuring the fluorescence of the sample at a plurality of different temperatures. For example, in some embodiments, the temperature of the sample in the fluorometer is heated to a target temperature. In some embodiments, the target temperature is about 60° C. or more, 70° C. or more, 80° C. or more, or about 90° C. or more. In some embodiments, the target temperature is about 100° C. or less, such as about 90° C. or less, about 80° C. or less, or about 70° C. or less. Fluorescence of the sample can be measured as the sample temperature is increasing and/or as the sample temperature is decreasing (for example, as the sample cools after reaching the target temperature). In some embodiments, the melting curve is generated after the isothermal amplification.

In some embodiments, the fluorometer is configured to detect fluorescence of the samples in the sample processing tubes from below the sample processing tubes. The sample processing tube holder of the fluorometer can include a clear or open bottom, so that a light source and an optical detector disposed below the sample processing tube holder can detect fluorescence emitted from the sample. The light source and/or optical detector can be movable to detect fluorescence from the sample processing tubes without moving the sample processing tubes. For example, the fluorometer can include a stepping motor, a guide shaft, towline, and/or timing belt to position the light source and/or detector underneath a sample processing tube before collecting fluorescence from that sample processing tube. In some embodiments, a light filter is included on the light source or light detector so that a narrowband light wavelength is detected by the detector or emitted from the light source. The wavelength of light emitted by the light source and detected by the detector is determined based on the fluorophores in the sample, and can be determined by a person of skill in the art. In some embodiments, the wavelength of light emitted by the light source or detected by the optical detector is about 200 nm to about 800 nm. In some embodiments, the fluorometer can emit or detect light at one or more (such as two or more, or three or more) different wavelengths. For example, in some embodiments the fluorometer can detect emission from a first fluorophore at a first wavelength and a second fluorophore at a second wavelength. The fluorometer can be connected to the computer system through a data port, and the detected fluorescence for a sample can be transmitted to the computer system.

The fluorometer includes a sample processing tube holder disposed above the light source that the detector, which is configured to receive the sample processing tubes. In some embodiments, the sample processing tubes are configured as a strip of linearly arranged connected sample processing tubes. In some embodiments, the sample processing tube holder includes a plurality of holes to receive the bottom ends of the sample processing tubes. The sample processing tube holder can include gaps or slots on adjacent to the holes, which allow the robotic arm to place or retrieve the strip.

FIG. 13 illustrates an exploded view of an exemplary fluorometer that can be used with the automated sample processing and analysis system. The fluorometer 1300 includes a sample processing tube holder 1302, which is configured to receive a plurality of sample processing tubes. In the illustrated example, the sample processing tube holder 1302 is configured to receive five strips comprising six linearly arranged connected sample processing tubes. The sample processing tube holder 1302 includes a plurality of slots 1304, and each slot can receive a strip of sample processing tubes. The slots include a plurality of holes 1306, and a sample processing tube can fit in each hole. The ends 1308 of the slots 1304 are extended, which allow the engagement region of the robotic arm to enter the slot 1304 to retrieve a strip in the slot 1304. The sample processing tube holder 1302 fits into a fluorometer lid 1310, which includes an opening 1312 to receive the sample processing tube holder 1302. Optionally, the fluorimeter lid 1310 includes a sample processing tube cooling rack 1314, which can receive one or more sample processing tubes. For example, in some embodiments, the robotic arm transports the one or more sample processing tubes (such as a strip) to the cooling rack 1314 after fluorescence has been measured at an elevated temperature. Once in the cooling rack, the sample processing tube can cool, which causes the wax (such as paraffin) in the sample processing tube to solidify. Once the wax is solidified, the robotic arm can transport the used sample processing tube to the solid waste management system. The fluorometer includes a reading module 1316 disposed below the sample processing tube holder 1302. The reading module 1316 includes the light source 1318 and the optical detector 1320. The reading module 1316 is configured to be movable in the fluorometer through the use of a first stepping motor 1322 and a second stepping motor 1324, which provide movement in the x-direction and y-direction. The reading module 1316 and the stepping motors 1322 and 1324 can be operated using the computer system, which is connected to the fluorometer 1300 through a data port 1326. The computer system can associate the location of the reading module 1316 (as controlled by the stepping motors 1322 and 1324) to a particular sample (which can be tracked throughout the automated system by the computer system) so that the detected fluoresce for a given sample is known.

The automated sample processing system described herein allows for high throughput processing and analysis of biological samples (such as blood, plasma, saliva, solid tissue, semen, sputum, or urine). During operation, components of the system are timed to minimize wait times so that a sample is not waiting for a downstream module to finish processing an earlier sample. In some embodiments, the automated system processes and analyzes samples at a rate of about 10 samples or more per hour, about 20 samples or more per hour, or about 30 samples or more per hour. Sample throughput is also benefited by using strips of connected sample processing tubes, which in some embodiments contains four to eight (such as six) sample processing tubes. By including a plurality of sample processing tubes in a strip, multiple samples can be simultaneously subjected to the same sample processing step. However, unlike high throughput systems that rely on large format multiwall plates (e.g., 48 well plates, 96 well plates, or larger formats), there is no need to wait to assemble a large number of samples before the sample processing steps begin. Further, in some embodiments, the system can be operated in an emergency setting, wherein a newly added sample is prioritized over samples that were already in the system.

The automated system for processing and analyzing the biological samples can include a computer system, which is configured to operate components of the system. For example, the computer system can include instructions for operating the robotic pipettor, the robotic arm, the fluorometer, the incubator, the shaker, the sample identification scanner, the nucleic acid isolation module, and/or any other processing or analyzing modules. In some embodiments, the computer system is configured to monitor an amount of consumables (e.g., sample processing tubes, pipette tips, and/or reagents) present in the system. For example, when an amount of a consumable is below a predetermined level or one or more components of the system have malfunctioned, the computer system can activate an indicator (such as a visual alarm, such as a light, or an audible alarm), which indicates an error in the system. In some embodiments, the indicator is a warning indicator, which indicates a potential upcoming error (such as consumables below a predetermined threshold or waste in the liquid or solid waste management system above a predetermined level). In some embodiments, the indicator is a stoppage indicator, indicating the system is out of one or more consumables or the liquid or waste management system is full. In some embodiments, if a stoppage indicator is triggered, the computer system automatically halts operation of the automated sample processing and analysis system.

In some embodiments, the computer systemtracks the location of one or more samples within the automated system. A sample source tube inputted into the system can include a sample identifier associated with the sample contained therein. The sample identifier scanner can scan the sample identifier at a known location (e.g., within the sample source tube holder), and the sample location can be communicated to the computer system by the sample identifier scanner. The computer system can then operate the robotic pipettor to transfer the sample to a sample processing tube at a known location. For example, a sample processing strip can be in a known bin location within the system, and the sample is transferred to a numbered sample processing tube within the strip (e.g., tube n of the strip x). The tube number and strip number of the sample can be recorded by the computer system. The target location of the sample (that is, which tube in which strip) can be dynamically determined based on the availability of unused sample processing tubes and/or unused strips, and the target location of a prior transferred sample. The computer system can also operate the robotic arm, which is used to transport the sample processing tubes throughout the system. Therefore, the computer system can track movement of the sample processing tube and the sample contained therein. Once the sample processing tube is moved to the fluorometer for analysis, the location of the sample within the fluorometer is known by the computer system, and determined fluorescence is associated with the sample.

In some embodiments, the computer system receives the fluorescence data generated by the fluorometer. In some embodiments, the data generated by the fluorometer is useful for quantitative “real time) nucleic acid amplification, such as an amplification curve or a melting curve. In some embodiments, the computer system includes a display and can display the fluorescence data on the display. The fluorescence data can include the time, temperature, and/or fluorescence of a sample. In some embodiments, the computer system displays a plot of the detected fluorescence against temperature (for example, to generate a melting curve), or a plot of the detected fluorescence against time (for example, to generate an amplification curve). In some embodiments, the computer system analyzes the data and can report or display a melting temperature (Tₘ), a number of copies of a region of interest, a cycle threshold (Ct) value, or any other suitable analytical output.

The computer system can be connected to a data network, and can transmit fluorescence data or analysis results across the data network. For example, in some embodiments the computer system is integrated with a laboratory information system (LIS), which may be operated by a hospital, clinician, pharmacy, or any other party. The data can be transmitted with a patient identifier (e.g., a name, a record number, a patient number, etc.) associated with the sample, which may be the same or different from the sample identifier. If the patient identifier is different from the sample identifier, the patient identifier and the sample identifier should be linked.

The computer system operates the robotic pipettor to withdraw and dispense liquids according to a predetermined workflow. Liquids can be withdrawn by the pipette at a first system component (such as the reagent rack or the sample source tube holder) and dispensed at a different system component (such as the one or more sample processing tube holders, the nucleic acid isolation system, or the fluorometer). In previous systems, occasional dripping from the pipette tip during the transfer of liquids from a first system component to a second system component could be a source of contamination. To minimize risk of contamination, in some embodiments, the movement path of the robotic pipette is predetermined. The pipette moves above the surface of the system, but passes over the system component from which the liquid is being withdrawn and the system component to which the liquid is being dispensed without passing over other system components. For example to transfer a reagent from the reagent rack to the a sample processing tube held in the nucleic acid isolation system, the robotic pipettor will withdraw the reagent from the reagent rack, move to the nucleic acid isolation system without passing over the shaker, incubator, or fluorometer, and dispense the reagent in the sample processing tube held by the nucleic acid isolation system. In another example, the robotic pipettor can withdraw liquids from the reagent rack and dispense the liquid in a sample processing tube held by the fluorometer without moving over the shaker, the heated incubator, or the nucleic acid isolation system.

The computer system can include a user interface (which may be a graphical user interface (GUI)), which can be displayed by the display. The user interface can be used to operate and/or monitor the automated system, such as by managing or reviewing sample inputs or data outputs, reviewing alerts or alarms, suspending or initiating the automated system, or controlling temperatures or incubation times.

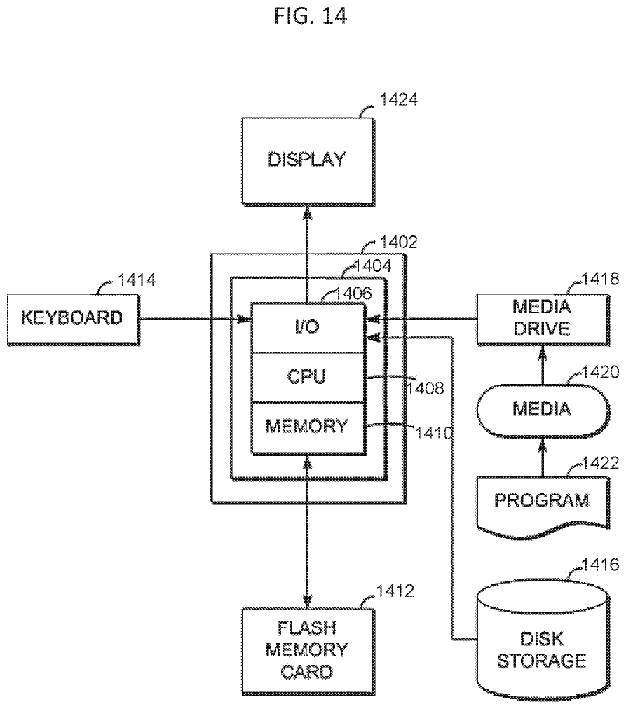

FIG. 14 depicts an exemplary computer system 1400 configured to perform any one of the processes described herein, including the various exemplary processes for operating the automated system, determining a melting curve, determining an amplification curve, or analyzing the melting curve or amplification curve. In this context, computing system 1400 may include, for example, a processor, non-transitory computer readable medium (e.g., memory), storage, and input/output devices (e.g., monitor, keyboard, disk drive, Internet connection, etc.). However, computing system 1400 may include circuitry or other specialized hardware for carrying out some or all aspects of the processes. In some operational settings, computing system 1400 may be configured as a system that includes one or more units, each of which is configured to carry out some aspects of the processes either in software, hardware, or some combination thereof.

FIG. 14 depicts computing system 1400 with a number of components that may be used to perform the above-described processes. The main system 1402 includes a motherboard 1404 having an input/output (“I/O”) section 1406, one or more central processing units (“CPU”) 1408, and a memory section 1410, which may have a flash memory card 1412 related to it. The I/O section 1406 is connected to a display 1424, a keyboard 1414, a disk storage unit 1416, and a media drive unit 1418. The media drive unit 1418 can read/write a computer-readable medium 1420, which can contain programs 1422 and/or data.

At least some values based on the results of the above-described processes can be saved for subsequent use. Additionally, a non-transitory computer-readable medium can be used to store (e.g., tangibly embody) one or more computer programs for performing any one of the above-described processes by means of a computer. The computer program may be written, for example, in a general-purpose programming language (e.g., Pascal, C, C++, Java, Python, JSON, etc.) or some specialized application-specific language.

In some embodiments, the automated nucleic acid isolation, amplification, and analysis system includes (i) a robotic pipettor comprising one or more pipettes movable in the a horizontal plane and configured to dispense or withdraw one or more liquids; (ii) a robotic arm configured to transport a plurality of connected sample processing tubes; (iii) a nucleic acid isolation system comprising a first sample processing tube holder configured to hold the plurality of connected sample processing tubes, and magnet; wherein magnetically-responsive particles contained within each sample processing tube are retained within the sample processing tube upon withdrawing of liquid by the robotic pipettor when the magnet is in an active configuration; and (iv) a fluorometer comprising a light source and an optical detector disposed below a second sample processing tube holder configured to hold the plurality of connected sample processing tubes, the second sample processing tube holder having a clear or open bottom, wherein the fluorometer is configured to detect fluorescence emitted from a sample in one or more of the sample processing tubes. In some embodiments, the fluorometer is configured to heat the plurality of connected sample processing tubes to a predetermined temperature above room temperature for nucleic acid amplification. In some embodiments, the system further includes one or more sample source tube holders configured to hold the plurality of sample source tubes, one or more heated incubators configured to heat the plurality of the connected sample processing tubes, and/or one or more shakers configured to vortex a sample contained within the sample processing tubes. In some embodiments, the system further comprises a barcode scanner configured to read a sample barcode disposed on one or more sample source tubes or the plurality of sample processing tubes. In some embodiments, the system further comprises a pipette tip holder accessible by the plurality of pipettes. In some embodiments, the system further comprises a reagent rack configured to hold one or more reagents.

In some embodiments, the automated nucleic acid isolation and analysis system includes (i) a robotic pipettor comprising one or more pipettes movable in the a horizontal plane and configured to dispense or withdraw one or more liquids; (ii) a robotic arm configured to transport a strip comprising a plurality of linearly arranged, connected sample processing tubes; (iii) a nucleic acid isolation system comprising a first sample processing tube holder configured to hold the plurality of connected sample processing tubes, and magnet; wherein magnetically-responsive particles contained within each sample processing tube are retained within the sample processing tube upon withdrawing of liquid by the robotic pipettor when the magnet is in an active configuration; and (iv) a fluorometer comprising a light source and an optical detector disposed below a second sample processing tube holder configured to hold the plurality of connected sample processing tubes, the second sample processing tube holder having a clear or open bottom, wherein the fluorometer is configured to detect fluorescence emitted from a sample in one or more of the sample processing tubes. In some embodiments, the fluorometer is configured to heat the strip to a predetermined temperature above room temperature for nucleic acid amplification. In some embodiments, the system further includes one or more sample source tube holders configured to hold the strip, one or more heated incubators configured to heat the strip, and/or one or more shakers configured to vortex a sample contained within the sample processing tubes of the strip. In some embodiments, the system further comprises a barcode scanner configured to read a sample barcode disposed on one or more sample source tubes or the strip. In some embodiments, the system further comprises a pipette tip holder accessible by the plurality of pipettes. In some embodiments, the system further comprises a reagent rack configured to hold one or more reagents.

In some embodiments, the automated nucleic acid isolation and analysis system includes (i) a robotic pipettor comprising one or more pipettes movable in the a horizontal plane and configured to dispense or withdraw one or more liquids; (ii) a robotic arm configured to transport a strip comprising a plurality of linearly arranged, connected sample processing tubes; (iii) a nucleic acid isolation system comprising a first sample processing tube holder configured to hold the plurality of connected sample processing tubes, and magnet; wherein magnetically-responsive particles contained within each sample processing tube are retained within the sample processing tube upon withdrawing of liquid by the robotic pipettor when the magnet is in an active configuration; (iv) heated vessel comprising wax, wherein the wax is heated by the vessel to a temperature above the melting temperature of the wax; and (v) a fluorometer comprising a light source and an optical detector disposed below a second sample processing tube holder configured to hold the plurality of connected sample processing tubes, the second sample processing tube holder having a clear or open bottom, wherein the fluorometer is configured to detect fluorescence emitted from a sample in one or more of the sample processing tubes. In some embodiments, the fluorometer is configured to heat the strip to a predetermined temperature above room temperature for nucleic acid amplification. In some embodiments, the system further includes one or more sample source tube holders configured to hold the strip, one or more heated incubators configured to heat the strip, and/or one or more shakers configured to vortex a sample contained within the sample processing tubes of the strip. in some embodiments, the system further comprises a barcode scanner configured to read a sample barcode disposed on one or more sample source tubes or the strip. In some embodiments, the system further comprises a pipette tip holder accessible by the plurality of pipettes. In some embodiments, the system further comprises a reagent rack configured to hold one or more reagents.

In some embodiments, the automated nucleic acid isolation and analysis system includes (i) a robotic pipettor comprising one or more pipettes movable in the a horizontal plane and configured to dispense or withdraw one or more liquids; (ii) a robotic arm configured to transport a strip comprising a plurality of linearly arranged, connected sample processing tubes; (iii) a nucleic acid isolation system comprising a first sample processing tube holder configured to hold the plurality of connected sample processing tubes, and magnet; wherein magnetically-responsive particles contained within each sample processing tube are retained within the sample processing tube upon withdrawing of liquid by the robotic pipettor when the magnet is in an active configuration; (iv) heated vessel comprising wax, wherein the wax is heated by the vessel to a temperature above the melting temperature of the wax; (v) a fluorometer comprising a light source and an optical detector disposed below a second sample processing tube holder configured to hold the plurality of connected sample processing tubes, the second sample processing tube holder having a clear or open bottom, wherein the fluorometer is configured to detect fluorescence emitted from a sample in one or more of the sample processing tubes; (vi) a liquid waste management system; and (vii) a solid waste management system. In some embodiments, the fluorometer is configured to heat the strip to a predetermined temperature above room temperature for nucleic acid amplification. In some embodiments, the system further includes one or more sample source tube holders configured to hold the strip, one or more heated incubators configured to heat the strip, and/or one or more shakers configured to vortex a sample contained within the sample processing tubes of the strip. In some embodiments, the system further comprises a barcode scanner configured to read a sample barcode disposed on one or more sample source tubes or the strip. In some embodiments, the system further comprises a pipette tip holder accessible by the plurality of pipettes. In some embodiments, the system further comprises a reagent rack configured to hold one or more reagents.

### Methods of Operating the Automated System

Samples loaded into the automated system are automatically processed and analyzed to collect fluorescence data, such as quantitative nucleic acid amplification data (e.g., an amplification curve) or a melting curve. The system is operated as a fully automated system without the need for user intervention other than to periodically refill reagents or consumables (such as wax or sample processing tubes), input or remove sample source tubes, and/or empty the solid or liquid waste management system. Once in operation, the system can operate continuously to process and analyze samples, with suspension occurring due to a lack of new samples or consumables or reagents.

In some aspects, the automated method for analyzing nucleic acid molecules in a sample includes isolating the nucleic acid molecules, combining the nucleic acid molecules with a nucleic acid probe and a fluorophore in a sample processing tube, measuring fluorescence of the sample, and discarding the sample processing tube. The nucleic acid molecules include a region of interest, and the nucleic acid probe binds to at least a portion of the region of interest. The method can be a multiplex method, wherein a plurality of different nucleic acid probes bind to different regions of interest in the nucleic acid molecule. Once the nucleic acid molecules in the sample have been isolated, the nucleic acid molecules are amplified and the sample is analyzed by fluorescence. In some embodiments, the sample is amplified and fluorescence measurements are taken simultaneously, for example to generate an amplification curve. In some embodiments, the sample is amplified and then fluorescence measurements are taken at a plurality of temperatures to generate a melting curve. In some embodiments, the method includes generating an amplification curve followed by generating a melting curve.

The nucleic acid molecules can be isolated, for example, by using the nucleic acid isolation system described herein. The sample can be transferred from a sample source tube to a sample processing tube, which may be part of a strip of sample processing tubes, by a robotic pipettor. The robotic pipettor adds magnetically-responsive particles to the sample processing tube. The magnetically-responsive particles are functionalized to bind nucleic acid molecules. The magnetically-responsive particles are in a suspension held by the reagent rack. The robotic pipettor withdraws a predetermined amount of the magnetically-responsive particles from the reagent rack and dispenses the particles in the sample processing tube. In some embodiments, one or more additional reagents may be added to the sample processing tube, such as saline or an internal, negative, or positive control. The sample processing tube can be held in a sample processing tube holder when the magnetically responsive particles, sample, and/or additional reagents are dispensed in the sample processing tube. Once the sample and magnetically-responsive particles are in the sample processing tube, the robotic arm can transport the sample processing tube to the shaker. The computer system can operate the shaker to vortex the sample contained in the sample processing tube. After the sample has been vortexed, the robotic arm can transport the sample processing tube to the heated incubator. The heated incubator can act to lyse cells in the sample and/or melt nucleic acid molecules in the sample. After incubation, the robotic arm transports the sample processing tube to a cooling rack. While the sample sits on the cooling rack, nucleic acid molecules in the sample can anneal to oligonucleotides or other binding agents on the magnetically-responsive particles so that the nucleic acid molecules are bound to the magnetically-responsive particles. After cooling, the robotic arm transports the sample processing tube to the nucleic acid isolation system. The nucleic acid isolation system includes a magnet, which interacts with the magnetically-responsive particles. The robotic pipettor removes liquid in the sample processing tube; however, since the magnetically-responsive particles interact with the magnet and the nucleic acid molecules are hound to the magnetically-responsive particles, the nucleic acid molecules remain in the sample processing tube. The liquid withdrawn from the sample processing tube can be dispensed in the liquid waste port of the liquid waste management system. In some embodiments, the nucleic acid molecules are washed with a wash buffer 1, 2 or more times. To wash the nucleic acid molecules, the robotic pipettor withdraws wash buffer from the reagent rack and dispenses the wash buffer in the sample processing tube. The robotic pipettor then withdraws the used wash buffer from the sample processing tube held in the nucleic acid isolation system and dispenses the used wash buffer in the liquid waste port of the liquid waste management system. In some embodiments, the robotic arm transports the sample processing tube to the sample processing tube holder (that is, removing it from the nucleic acid isolation system) when the wash buffer is added to the sample processing tube. In some embodiments, the sample processing tube is transported by the robotic arm to the shaker and vortexed after the wash buffer has been added to the sample processing tube before the sample processing tube is returned to the nucleic acid isolation system to withdraw the used wash buffer.

After the nucleic acid molecules are isolated (which optionally includes one or more washing steps), the sample processing tube is transported to the sample processing tube holder by the robotic arm. The robotic pipettor withdraws amplification reagents (such as nucleotides, buffers, nucleic acid probes, enzyme, fluorophores, etc.) and dispenses the amplification reagents into the sample processing tube holder. The sample processing tube with the amplification reagents are then transported to the heated incubator by the robotic arm, which allows the nucleic acid molecules to melt. In some embodiments, the robotic arm transports the sample processing tube to the shaker and the sample is vortexed prior to the robotic arm transporting the sample processing tube to the heated incubator. In some embodiments, the robotic pipettor withdraws melted wax (e.g., melted paraffin) from the heated vessel and dispenses the melted wax in the sample processing tube.

After incubation in the heated incubator, the robotic arm transports the sample processing tube to the fluorometer for analysis. In some embodiments, the fluorometer is pre-heated for isothermal amplification. The isothermal amplification temperature is above room temperature, such as between about 30° C. and about 80° C. (such as between about 30° C. and about 60° C., between about 37° C. and about 47° C., or about 42° C.). The robotic pipettor can withdraw amplification enzyme form the reagent rack and dispense the enzyme into the sample processing tube. The nucleic acid molecules in the sample processing tube are then amplified by isothermal amplification. Simultaneously, the fluorescence is measured by the fluorometer, and the fluorescence data for the sample is transmitted to the computer system. In some embodiments, amplification is performed by isothermal nucleic acid amplification, fir example as described in International Patent Application Publication WO 2011/091393 A2, hereby incorporated herein by reference in its entirety. As the number of amplicons in the sample processing tube increases, detected fluorescence increases. The fluorescence can be measured as a function of time during the isothermal amplification to obtain and amplification curve. In some embodiments, the fluorescence is measured every 10 minutes or more frequently, every 5 minutes or more frequently, every 3 minutes or more frequently, every 2 minutes or more frequently, every minute or more frequently, or every 30 seconds or more frequently. In some embodiments, isothermal amplification proceeds for about 20 minutes or more (such as about 30 minutes or more, or about 40 minutes or more). The measured fluorescence for the sample can be transmitted to the computer system.

In some embodiments, after amplification of the nucleic acid sample, a melting curve is generated. The temperature of the sample in the fluorometer is increased to a target temperature. In some embodiments, the target temperature is about 60° C. or more, 70° C. or more, 80° C. or more, or about 90° C. or more. In some embodiments, the target temperature is about 100° C. or less, such as about 90° C. or less, about 80° C. or less, or about 70° C. or less. Fluorescence of the sample is measured as the sample temperature is increasing and/or as the sample temperature is decreasing (for example, as the sample cools after reaching the target temperature). The measured fluorescence for the sample and the temperature of the fluorescence measurement can be transmitted to the computer system.

After the fluorescence data of the sample is measured by the fluorometer, the robotic arm can remove the sample processing tube from the fluorometer. In some embodiments, the robotic arm moves the sample processing tube to the solid waste management system for disposal. In some embodiments, the robotic arm moves the sample processing tube from the fluorometer to a sample processing tube holder to cool. During the cooling process, wax in the sample processing tube (if added) can solidify, thereby sealing liquids in the sample processing tube. Once the wax is solidified, the robotic arm moves the sample processing tube to the solid waste management system for disposal.

In some embodiments, the method of analyzing nucleic acid molecules in the sample includes (i) isolating nucleic acid molecules comprising a region of interest from the sample; (ii) combining the nucleic acid molecules, a nucleic acid probe that hybridizes to at least a portion of the region of interest, and a fluorophore; (iii) adding a melted wax with a melting temperature above room temperature (such as paraffin) to a sample processing tube containing the sample; (iv) amplifying the region of interest (such as by isothermal amplification); (v) measuring fluorescence of the combined region of interest, the nucleic acid detection probe, and the fluorophore; (vi) solidifying the wax in the sample processing tube; and (vii) discarding the sample processing tube comprising the solidified wax. In some embodiments, the fluorophore is attached to the nuclear probe. In some embodiments, amplifying and measuring fluorescence occur simultaneously. In some embodiments, the fluorescence is measured from below the sample processing tube. In some embodiments, isolating the nucleic acid comprises binding magnetically-responsive particles functionalized with a nucleic acid capture probe to the nucleic acid molecules comprising the region of interest. In some embodiments, isolating the nucleic acid molecules comprises lysing cells containing the nucleic acid molecules. In some embodiments, the method comprises washing the magnetically-responsive particles bound to the nucleic acid molecules comprising the region of interest. In some embodiments, isolating the nucleic acid molecules comprises lysing cells containing the nucleic acid molecules.

In some embodiments, the method of analyzing nucleic acid molecules in the sample includes (i) isolating nucleic acid molecules comprising a region of interest from the sample; (ii) combining the nucleic acid molecules, a nucleic acid probe that hybridizes to at least a portion of the region of interest, and a fluorophore; (iii) adding a melted wax with a melting temperature above room temperature (such as paraffin) to a sample processing tube containing the sample; (iv) amplifying the region of interest (such as by isothermal amplification); (v) measuring fluorescence of the combined region of interest, the nucleic acid detection probe, and the fluorophore; (vi) solidifying the wax in the sample processing tube; (vii) discarding the sample processing tube comprising the solidified wax; and (viii) determining an amplification curve. In some embodiments, the fluorophore is attached to the nuclear probe. In some embodiments, amplifying and measuring fluorescence occur simultaneously. In some embodiments, the fluorescence is measured from below the sample processing tube. In some embodiments, isolating the nucleic acid comprises binding magnetically-responsive particles functionalized with a nucleic acid capture probe to the nucleic acid molecules comprising the region of interest. In some embodiments, isolating the nucleic acid molecules comprises lysing cells containing the nucleic acid molecules. In some embodiments, the method comprises washing the magnetically-responsive particles bound to the nucleic acid molecules comprising the region of interest. In some embodiments, isolating the nucleic acid molecules comprises lysing cells containing the nucleic acid molecules.

In some embodiments, the method of analyzing nucleic acid molecules in the sample includes (i) isolating nucleic acid molecules comprising a region of interest from the sample; (ii) combining the nucleic acid molecules, a nucleic acid probe that hybridizes to at least a portion of the region of interest, and a fluorophore; (iii) adding a melted wax with a melting temperature above room temperature (such as paraffin) to a sample processing tube containing the sample; (iv) amplifying the region of interest (such as by isothermal amplification); (v) measuring fluorescence of the combined region of interest, the nucleic acid detection probe, and the fluorophore; (vi) solidifying the wax in the sample processing tube; (vii) discarding the sample processing tube comprising the solidified wax; (viii) determining an amplification curve; and (ix) determining a melting curve. In some embodiments, the fluorophore is attached to the nuclear probe. In some embodiments, amplifying and measuring fluorescence occur simultaneously. In some embodiments, the fluorescence is measured from below the sample processing tube. In some embodiments, isolating the nucleic acid comprises binding magnetically-responsive particles functionalized with a nucleic acid capture probe to the nucleic acid molecules comprising the region of interest. In some embodiments, isolating the nucleic acid molecules comprises lysing cells containing the nucleic acid molecules. In some embodiments, the method comprises washing the magnetically-responsive particles bound to the nucleic acid molecules comprising the region of interest. In some embodiments, isolating the nucleic acid molecules comprises lysing cells containing the nucleic acid molecules.

In some embodiments, the method of analyzing nucleic acid molecules in the sample includes (i) isolating nucleic acid molecules comprising a region of interest from the sample; (ii) combining the nucleic acid molecules, a nucleic acid probe that hybridizes to at least a portion of the region of interest, and a fluorophore; (iii) adding a melted wax with a melting temperature above room temperature (such as paraffin) to a sample processing tube containing the sample; (iv) amplifying the region of interest (such as by isothermal amplification); (v) measuring fluorescence of the combined region of interest, the nucleic acid detection probe, and the fluorophore; (vi) solidifying the wax in the sample processing tube; (vii) discarding the sample processing tube comprising the solidified wax; and (viii) determining an amplification curve; wherein the method is performed by an automated system. In some embodiments, the fluorophore is attached to the nuclear probe. In some embodiments, amplifying and measuring fluorescence occur simultaneously. In some embodiments, the fluorescence is measured from below the sample processing tube. In some embodiments, isolating the nucleic acid comprises binding magnetically-responsive particles functionalized with a nucleic acid capture probe to the nucleic acid molecules comprising the region of interest. In some embodiments, isolating the nucleic acid molecules comprises lysing cells containing the nucleic acid molecules. In some embodiments, the method comprises washing the magnetically-responsive particles bound to the nucleic acid molecules comprising the region of interest. In some embodiments, isolating the nucleic acid molecules comprises lysing cells containing the nucleic acid molecules.

In some embodiments, the method of analyzing nucleic acid molecules in the sample includes (i) isolating nucleic acid molecules comprising a region of interest from the sample; (ii) combining the nucleic acid molecules, a nucleic acid probe that hybridizes to at least a portion of the region of interest, and a fluorophore; (iii) adding a melted wax with a melting temperature above room temperature (such as paraffin) to a sample processing tube containing the sample; (iv) amplifying the region of interest (such as by isothermal amplification); (v) measuring fluorescence of the combined region of interest, the nucleic acid detection probe, and the fluorophore; (vi) solidifying the wax in the sample processing tube; (vii) discarding the sample processing tube comprising the solidified wax; determining an amplification curve; and (ix) determining a melting curve; wherein the method is performed by an automated system. In some embodiments, the fluorophore is attached to the nuclear probe. some embodiments, amplifying and measuring fluorescence occur simultaneously. In some embodiments, the fluorescence is measured from below the sample processing tube. In some embodiments, isolating the nucleic acid comprises binding magnetically-responsive particles functionalized with a nucleic acid capture probe to the nucleic acid molecules comprising the region of interest. In some embodiments, isolating the nucleic acid molecules comprises lysing cells containing the nucleic acid molecules. In some embodiments, the method comprises washing the magnetically-responsive particles bound to the nucleic acid molecules comprising the region of interest. In some embodiments, isolating the nucleic acid molecules comprises lysing cells containing the nucleic acid molecules.

In some embodiments, a method of determining a melting curve of a nucleic acid sample includes (i) isolating nucleic acid molecules comprising a region of interest from the sample; (ii) combining the nucleic acid molecules, a nucleic acid probe that hybridizes to at least a portion of the region of interest, and a fluorophore; (iii) adding a melted wax with a melting temperature above room temperature (such as paraffin) to a sample processing tube containing the sample; (iii) amplifying the region of interest from the nucleic acid molecules; (iv) measuring fluorescence of the combined region of interest, the nucleic acid detection probe, and fluorophore at a plurality of temperatures; (v) solidifying the wax contained within the sample processing tube; and (vi) discarding the sample processing tube comprising the solidified wax. In some embodiments, the fluorophore is attached to the nuclear probe. In some embodiments, amplifying and measuring fluorescence occur simultaneously. In some embodiments, the fluorescence is measured from below the sample processing tube. In some embodiments, isolating the nucleic acid comprises binding magnetically-responsive particles functionalized with a nucleic acid capture probe to the nucleic acid molecules comprising the region of interest. In some embodiments, isolating the nucleic acid molecules comprises lysing cells containing the nucleic acid molecules. In some embodiments, the method comprises washing the magnetically-responsive particles bound to the nucleic acid molecules comprising the region of interest. In some embodiments, isolating the nucleic acid molecules comprises lysing cells containing the nucleic acid molecules.

In some embodiments, a method of determining a melting curve of a nucleic acid sample includes (i) isolating nucleic acid molecules comprising a region of interest from the sample; (ii) combining the nucleic acid molecules, a nucleic acid probe that hybridizes to at least a portion of the region of interest, and a fluorophore; (iii) adding a melted wax with a melting temperature above room temperature (such as paraffin) to a sample processing tube containing the sample; (iii) amplifying the region of interest from the nucleic acid molecules; (iv) measuring fluorescence of the combined region of interest, the nucleic acid detection probe, and fluorophore at a plurality of temperatures; (v) solidifying the wax contained within the sample processing tube; and (vi) discarding the sample processing tube comprising the solidified wax; wherein the method is performed by an automated system. In some embodiments, the fluorophore is attached to the nuclear probe. In some embodiments, amplifying and measuring fluorescence occur simultaneously. In some embodiments, the fluorescence is measured from below the sample processing tube. In some embodiments, isolating the nucleic acid comprises binding magnetically-responsive particles functionalized with a nucleic acid capture probe to the nucleic acid molecules comprising the region of interest. In some embodiments, isolating the nucleic acid molecules comprises lysing cells containing the nucleic acid molecules. In some embodiments, the method comprises washing the magnetically-responsive particles bound to the nucleic acid molecules comprising the region of interest. In some embodiments, isolating the nucleic acid molecules comprises lysing cells containing the nucleic acid molecules.

In some embodiments, a method of analyzing a nucleic acid sample includes (i) dispensing a sample comprising nucleic acid molecules comprising a region of interest from a sample source container into a sample processing tube selected from a plurality of connected sample processing tubes; (ii) combining the sample with magnetically-responsive particles functionalized with a probe that binds to the nucleic acid molecules comprising the region of interest; (iii) transporting the sample processing tube using a robotic arm to a magnetic module comprising a first sample holder configured to hold the plurality of connected sample processing tubes, and magnet; (iv) washing the nucleic acid molecules using a robotic pipettor by dispensing and withdrawing a wash buffer into the sample processing tube, wherein the magnet is in an active configuration when the wash buffer is withdrawn, thereby retaining the magnetically-responsive particles in the sample processing tube; (v) adding a melted wax (such as paraffin) the sample processing tube using the robotic pipettor, wherein the wax has a melting temperature above room temperature to the sample processing tube; (vi) adding an amplification reagent, a nucleic acid probe that specifically binds to the nucleic acid molecules, and a fluorophore to the sample processing tube using the robotic pipettor; (vii) transporting the sample processing tube to a second sample holder on a fluorometer using the robotic arm, wherein the second sample holder is disposed above a light source and an optical detector; (viii) simultaneously heating the sample processing tube and detecting fluorescence from the sample; (ix) cooling the sample processing tube, thereby solidifying the wax; and (x) discarding the sample processing tube comprising the solidified wax. In some embodiments, the fluorophore is attached to the nuclear probe. In some embodiments, amplifying and measuring fluorescence occur simultaneously. In some embodiments, the fluorescence is measured from below the sample processing tube. In some embodiments, isolating the nucleic acid comprises binding magnetically-responsive particles functionalized with a nucleic acid capture probe to the nucleic acid molecules comprising the region of interest. In some embodiments, isolating the nucleic acid molecules comprises lysing cells containing the nucleic acid molecules. In some embodiments, the method comprises washing the magnetically-responsive particles bound to the nucleic acid molecules comprising the region of interest. In some embodiments, isolating the nucleic acid molecules comprises lysing cells containing the nucleic acid molecules.

In some embodiments, the automated system described herein is operated to perform one or more methods described in WO2011/0091393. For example, in one aspect, there is a method for selective amplification of a target polynucleotide sequence, comprising: (a) combining the target polynucleotide sequence with a first composite primer in a sample processing tube using a robotic pipettor comprising one or more pipettes movable in a horizontal plane to hybridize the target polynucleotide sequence with the first composite primer, said first composite primer comprising a 5′ promotor portion (P) and a 3′ target-recognition portion which is complementary to the 3′ end of the target polynucleotide sequence; (b) adding a fluorophore to the sample processing tube using the robotic pipettor, (c) incubating the sample processing tube, thereby (1) extending the 3′ end of the first composite primer and generating a first single stranded nucleic acid (e.g., DNA) template comprising the promoter portion (P) and the complementary sequence of the target polynucleotide sequence (Tc), said first single-stranded nucleic acid (e.g., DNA) template comprising a first pair of self-folding segments that are complementary to each other, wherein the promotor portion (P) is at the 5′ end of the first single-stranded nucleic acid (e.g., DNA) template and one of the self-folding segments is at the 3′ end of the first single-stranded nucleic acid (e.g., DNA) template, (2) allowing the first single-stranded nucleic acid (e.g., DNA) template to self-fold and form a first handle-step-loop structure, which comprises a 5′ single-stranded handle comprising the promotor portion (P) and a double-stranded stem comprising the first pair of self-folding segments hybridized to each other, (3) extending the 3′ end of the first handle-stem-loop structure to generate a double-stranded promoter comprising the promotor portion (P) and its complementary sequence (Pc) hybridized to each other, and (4) transcribing from the double-stranded promotor to generate multiple copies of a single-stranded RNA product comprising the target polynucleotide sequence. In some embodiments, the first composite primer comprises a first self-folding segment between the promotor portion and the 3′ target recognition portion.

In another aspect, there is a method for selective amplification of a target polynucleotide sequence, comprising: (a) combining the target polynucleotide sequence with a first composite primer in a sample processing tube using a robotic pipettor comprising one or more pipettes movable in a horizontal plane to hybridize the target polynucleotide sequence with the first composite primer, said first composite primer comprising a 5′ promotor portion (P) and a 3′ target-recognition portion which is complementary to the 3′ end of the target polynucleotide sequence; (b) adding a fluorophore to the sample processing tube using the robotic pipettor, (c) adding a melted wax with a melting temperature above room temperature to the sample processing tube, and (d) incubating the sample processing tube, thereby (1) extending the 3′ end of the first composite primer and generating a first single stranded nucleic acid (e.g., DNA) template comprising the promoter portion (P) and the complementary sequence of the target polynucleotide sequence (Tc), said first single-stranded nucleic acid (e.g., DNA) template comprising a first pair of self-folding segments that are complementary to each other, wherein the promotor portion (P) is at the 5′ end of the first single-stranded nucleic acid (e.g., DNA) template and one of the self-folding segments is at the 3′ end of the first single-stranded nucleic acid (e.g., DNA) template, (2) allowing the first single-stranded nucleic acid (e.g., DNA) template to self-fold and form a first handle-step-loop structure, which comprises a 5′ single-stranded handle comprising the promotor portion (P) and a double-stranded stem comprising the first pair of self-folding segments hybridized to each other, (3) extending the 3′ end of the first handle-stein-loop structure to generate a double-stranded promoter comprising the promotor portion (P) and its complementary sequence (Pc) hybridized to each other, and (4) transcribing from the double-stranded promotor to generate multiple copies of a single-stranded RNA product comprising the target polynucleotide sequence. In some embodiments, the first composite primer comprises a first self-folding segment between the promotor portion and the 3′ target recognition portion.

Various exemplary embodiments are described herein. Reference is made to these examples in a non-limiting sense. They are provided to illustrate more broadly applicable aspects of the disclosed technology. Various changes may be made and equivalents may be substituted without departing from the true spirit and scope of the various embodiments. In addition, many modifications may be made to adapt a particular situation, material, composition of matter, process, process act(s) or step(s) to the objective(s), spirit or scope of the various embodiments. Further, as will be appreciated by those with skill in the art, each of the individual variations described and illustrated herein has discrete components and features that may be readily separated from or combined with the features of any of the other several embodiments without departing from the scope or spirit of the various embodiments, All such modifications are intended to be within the scope of claims associated with this disclosure.

What is claimed is:

1. An automated nucleic acid isolation and analysis system, comprising:
a robotic pipettor comprising one or more pipettes movable in a horizontal plane and configured to dispense or withdraw one or more liquids;
a robotic arm configured to transport a plurality of connected sample processing tubes;
a nucleic acid isolation system comprising a first sample processing tube holder configured to hold the plurality of connected sample processing tubes, and magnet; wherein magnetically-responsive particles contained within each sample processing tube are retained within the sample processing tube upon withdrawing of liquid by the robotic pipettor when the magnet is in an active configuration; and
a fluorometer comprising a light source and an optical detector disposed below a second sample processing tube holder configured to hold the plurality of connected sample processing tubes, the second sample processing tube holder having a clear or open bottom, wherein the fluorometer is configured to detect fluorescence emitted from a sample in one or more of the sample processing tubes.

2. The system of claim 1, wherein the fluorometer is configured to heat the plurality of connected sample processing tubes to a predetermined temperature above room temperature.

3. The system of claim 1 or 2, wherein the plurality of connected sample processing tubes is a sample strip comprising three or more linearly arranged sample processing tubes.

4. The system of claim 1 or 2, wherein the plurality of connected sample processing tubes is a multi-well plate.

5. The system of any one of claims 1-4, comprising a heated vessel comprising wax, wherein the wax is heated by the vessel to a temperature above the melting temperature of the wax.

6. The system of any one of claims 1-5, comprising one or more heated incubators configured to heat the plurality of connected sample processing tubes.

7. The system of any one of claims 1-6, comprising one or more shakers configured to vortex a sample contained within the sample processing tubes.

8. The system of any one of claims 1-7, comprising a sample source tube holder configured to hold the plurality of sample source tubes.

9. The system of claim 8, wherein the system comprises a barcode scanner configured to read a sample barcode disposed on one or more sample source tubes or the plurality of sample processing tubes.

10. The system of any one of claims 1-9, comprising a pipette tip holder accessible by the plurality of pipettes.

11. The system of any one of claims 1-10, comprising a reagent rack configured to hold one or more reagents.

12. The system of any one of claim 1-11, comprising a solid waste management system configured to receive pipette tips and the plurality of sample processing tubes.

13. The system of any one of claims 1-2, comprising one of more cooling racks configured to hold the sample processing tube.

14. The system of any one of claims 1-13, comprising a liquid waste management system comprising a liquid waste port and a conduit configured to drain the liquid waste from the liquid waste port.

15. The system of any one of claims 1-14, wherein the robotic pipettor is operable to move the plurality of pipettes in a predetermined path that prevents the plurality of pipettes from moving above a non-targeted system component.

16. The system of any one of claims 1-15, comprising a housing enclosing the system, the housing comprising a base and an openable lid.

17. The system of claim 16, wherein the housing comprises a ventilation system comprising an air filter, wherein the ventilation system is configured to provide filtered air to the enclosed system and withdraw air from the enclosed system.

18. The system of claim 16 or 17, wherein the enclosed system is operated at a higher pressure than the pressure external to the housing.

19. The system of any one of claims 16-18, wherein the housing comprises one or more indicator lights on an external surface of the housing configured to indicate normal operation of the system or an error.

20. The system of any one of claims 16-19, comprising a UV light within the housing configured to sterilize the system when the UV light is operated.

21. The system of any one of claims 1-20, wherein the system comprises an indicator o indicate an error.

22. The system of claim 21, wherein the indicator is a light or an audible alarm.

23. The system of any one of claims 1-22, comprising a computer system for operating the automated nucleic acid isolation and analysis system.

24. The system of claim 23, wherein the computer system comprises a display.

25. The system of claim 2.3 or 24, wherein the computer system is connected to a laboratory information system configured to store or transmit sample analysis results.

26. A method of analyzing nucleic acid molecules in a sample, comprising:
isolating nucleic acid molecules comprising a region of interest from the sample;
combining the nucleic acid molecules, a nucleic acid probe that hybridizes to at least a portion of the region of interest, and a fluorophore;
adding a melted wax with a melting temperature above room temperature to a sample processing tube containing the sample;
amplifying the region of interest;
measuring fluorescence of the combined region of interest, the nucleic acid detection probe, and the fluorophore;
solidifying the wax in the sample processing tube; and
discarding the sample processing tube comprising the solidified wax.

27. The method of claim 26, comprising determining an amplification curve for the sample.

28. The method of claim 26 or 27, wherein the fluorophore is attached to the nucleic acid probe.

29. The method of claim 26 or 27, wherein the fluorophore is separate from the nucleic acid probe.

30. A method of determining a melting curve of a nucleic acid sample, comprising isolating nucleic acid molecules comprising a region of interest from the sample;
combining the nucleic acid molecules, a nucleic acid probe that hybridizes to at least a portion of the region of interest, and a fluorophore;
adding a melted wax with a melting temperature above room temperature to a sample processing tube containing the sample;
amplifying the region of interest from the nucleic acid molecules;
measuring fluorescence of the combined region of interest, the nucleic acid detection probe, and fluorophore at a plurality of temperatures;
solidifying the wax contained within the sample processing tube; and
discarding the sample processing tube comprising the solidified wax.

31. The method of claim 30, wherein the fluorophore is separate from the nucleic acid probe.

32. The method of any one of claims 26-31, wherein the method is performed by an automated system.

33. The method of any one of s 26-32, wherein the sample processing tube is passively cooled.

34. The method of any one of claims 26-33, comprising heating the sample processing tube to denature the region of interest and the nucleic acid detection probe.

35. The method of any one of claims 26-34, wherein isolating the nucleic acid comprises binding magnetically-responsive particles functionalized with a nucleic acid capture probe to the nucleic acid molecules comprising the region of interest.

36. The method of claim 35. comprising washing the magnetically-responsive particles bound to the nucleic acid molecules comprising the region of interest.

37. The method of any one of claims 26-36, wherein isolating the nucleic acid molecules comprises lysing cells containing the nucleic acid molecules.

38. The method of any one of claims 26-37, wherein fluorescence is measured from below the sample processing tube.

39. The method of any one of claims 26-38, further comprising analyzing the measured fluorescence to determine an amount of the region of interest in the sample.

40. The method of any one of claims 26-39, wherein the melting temperature the wax is about 30° C. to about 90° C.

41. The method of any one of claims 26-40, wherein the wax is paraffin.

42. A method of analyzing a nucleic acid sample, comprising:
dispensing a sample comprising nucleic acid molecules comprising a region of interest into a sample processing tube selected from a plurality of connected sample processing tubes;
combining the sample with magnetically-responsive particles functionalized with a probe that binds to the nucleic acid molecules comprising the region of interest;
transporting the sample processing tube using a robotic arm to a magnetic module comprising a first sample holder configured to hold the plurality of connected sample processing tubes, and magnet;
washing the nucleic acid molecules using a robotic pipettor by dispensing and withdrawing a wash buffer into the sample processing tube, wherein the magnet is in an active configuration when the wash buffer is withdrawn, thereby retaining the magnetically-responsive particles in the sample processing tube;
adding a melted wax the sample processing tube using the robotic pipettor, wherein the wax has a melting temperature above room temperature to the sample processing tube;
adding an amplification reagent, a nucleic acid probe that specifically binds to the nucleic acid molecules, and a fluorophore to the sample processing tube using the robotic pipettor;
transporting the sample processing tube to a second sample holder on a fluorometer using the robotic arm, wherein the second sample holder is disposed above a light source and an optical detector;
simultaneously heating the sample processing, tube and detecting fluorescence from the sample;
cooling the sample processing tube, thereby solidifying the wax; and
discarding the sample processing tube comprising the solidified wax.
